(19) United States
(12) Patent Application Publication
(10) Pub. No.: US 2015/0153356 A1
(43) Pub. Date: Jun. 4, 2015

(54) PEPTIDE AND ANTIBODY LIBRARIES AND USES THEREOF

(71) Applicant: AbMART (SHANGHAI) Co., Ltd., Shanghai (CN)

(72) Inventors: Xun Meng, Shanghai (CN); Yang Li, Shanghai (CN); Xiaoqing Wang, Shanghai (CN)

(73) Assignee: AbMART (SHANGHAI) CO., LTD., Shanghai (CN)

(21) Appl. No.: 14/388,907

(22) PCT Filed: Apr. 1, 2013
(86) PCT No.: PCT/CN2013/073571

§ 371 Date: Sep. 29, 2014

(51) Int. Cl. G01N 33/68; C12N 5/16

(52) U.S. Cl. CPC G01N 33/6854; C12N 5/163; G01N 2333/91215; G01N 2333/9121; G01N 2333/4712; G01N 2333/28; G01N 2333/415; G01N 2333/475

(57) ABSTRACT

This invention relates generally to antibody libraries, polypeptide libraries and the uses thereof, including methods for producing antibody libraries, methods for identifying an antibody to a target, and methods for identifying a target associated with a condition. In specific embodiments, the present invention relates to an antibody library comprising at least 10,000 different members, which can be used for screening an antibody with high affinity against a protein of interest

### CROSS-REFERENCE TO RELATED APPLICATIONS

This application claims benefit of priority of PCT Application No. PCT/CN2012/000430, filed on Mar. 31, 2012, which is hereby incorporated by reference in its entirety.

### FIELD OF THE INVENTION

This invention relates generally to antibodies, antibody libraries, polypeptides, polypeptide libraries and the uses thereof, including methods for producing antibody libraries, methods for identifying an antibody to a target, and methods for identifying a target associated with a condition. In specific embodiments, the present invention relates to an antibody library comprising at least 10,000 different members, which can be used for screening an antibody with high affinity against a target, e.g., a protein, of interest.

### BACKGROUND OF THE INVENTION

In the fields of medicine, biology, pharmacy etc., the demand for various antibodies and antibody libraries is rapidly increasing. Currently, the commonly used methods for obtaining antibodies include hybridoma techniques, recombinant antibody techniques, various molecular display techniques, and those techniques combining the above techniques with high-throughput processes, etc.

In conventional antibody preparation techniques, it will generally be necessary to immunize an animal using a native or recombinant protein or fragment thereof, so as to allow the animal to produce an antibody that can specifically recognize and bind said protein. And the antibody can then be obtained from cells of the animal through various technical means according to different requirements.

The obtaining of an antibody can involve hybridoma techniques. In such processes, after immunizing the animal, it will be necessary to take animal cells and then conduct cell fusion, so as to obtain the hybridoma that produces antibody; the hybridoma is then cloned and established as a strain to produce antibodies, and subsequently the antibodies are purified and identified. According to the requirements, the epitope of the antigen can also be determined. The detailed procedure of such hybridoma techniques can be found in various textbooks and manuals (such as Bazin, Rat hybridomas and rat monoclonal antibodies, CRC Press, 1990; Goding, Monoclonal antibodies: principles and practice, 3^{rd} edition, Academic Press, 1996; Shepherd and Dean Monoclonal antibodies, Oxford University Press, 2000 etc). Currently these processes are still widely employed for antibody preparation, but they have many disadvantages, such as long preparation period, very complicated preparation techniques, and high cost. Furthermore, these processes cannot be applied to all the proteins, e.g., for some antigens with poor solubility, low immunogenicity, or toxicity, these processes are not applicable (Sinclair N R et al., 2004 B cell/antibody tolerance to our own antigens. Front Biosci 9: 3019-3028).

Additionally, in order to obtain monoclonal antibodies with specificity, the commonly used way is to couple the chemically synthesized peptide fragment into a carrier protein, and then use it to immunize a mouse. Such process can produce antibodies against a single epitope of the same protein. However, due to the difference in the immunogenicity of various peptide fragments, the overall success rate of such strategy is not good enough, especially for proteins showing high homology to the host, the peptides thereof will have very poor immunogenicity, and can hardly stimulate potent immune reaction in mouse. Another conventional strategy is using full length protein or fragment thereof as immunogen, this strategy partially solved the above mentioned problem, but there still exists some other defects, such as low overall success rate in protein expression (30-70% for normal expression and purification systems) (Thorsten Kohl, Christian Schmidt, Stefan Wiemann, Annemarie Poustka and Ulrike Korf. Drew, 2003). As for protein fragments with high homology to the host animal used, their immune response in the animal is normally weak, and the success rate for preparing monoclonal antibody is relatively low (Sinclair N R et al, 2004, Automated production of recombinant human proteins as resource for proteome research, Proteome Science 2008, 6:4; Sinclair N R (2004) B cell/antibody tolerance to our own antigens. Front Biosci 9: 3019-3028).

The recombinant antibody techniques can be combined with various molecular display techniques, so as to produce antibodies (with high affinity to the targets) against multiple antigens, epitopes of the antigens can be simultaneously determined, and thus this strategy is commonly used in drug development (Christine Rothe, Stefanie Urlinger, Makiko Yamashita et al. The Human Combinatorial Antibody Library HuCAL GOLD Combines Diversification of All Six CDRs According to the Natural Immune System with a Novel Display Method for Efficient Selection of High-Affinity Antibodies. J. Mol. Biol. (2008) 376, 1182-1200, 2007). But the operation of recombinant antibody technique is complicated, costly, the yield thereof is relatively low, and there often exists unspecific binding, such that these techniques cannot be widely used (Levitan, B. Stochastic modeling and optimization of phage display. J. Mol. Biol. 277, 893-916 (1998). Bradbury et al, 2004).

As such, all the conventional antibody preparation methods in the art typically involve complicated operational processes, such as immunizing animals, preparing hybridoma cells etc. Therefore they all have the disadvantages of long preparation period, complicated preparation techniques, and high cost etc.

Antibody library technique based on phage antibodies is a novel antibody preparation technique developed recently. The antibody gene fragments cloned in vitro are inserted into phage vectors, and are then expressed; subsequently, antigens are used to perform screening against the expressed antibody library, and thereby monoclonal phage antibodies with specificity are obtained. However, phage antibody library technique will need extremely large amount of antibodies in the library so as to obtain antibody with high affinity, and the antibody production procedure is still relatively complicated.

In order to solve the above problems, a novel method for preparing or screening antibodies is desired, so as to omit the complicated, time-consuming, and costly processes, and thus antibodies with high affinity can be rapidly and effectively prepared or screened. The present invention addresses the above and other related concerns in the art.

### BRIEF SUMMARY OF THE INVENTION

In one aspect, the present invention is directed to a method for identifying an antibody to a target, which method comprises: a) providing for an antibody library obtained from a subject, e.g., a mammal, wherein said antibody library comprises less than 10⁷ different kinds of antibodies; b) contacting said target with said antibody library under conditions suitable for binding between said target with an antibody in said antibody library, if such antibody existing in said antibody library; and c) assessing binding between said target and said antibody to identify said antibody as an antibody to said target. In some embodiments, the antibody library is obtained from a subject or mammal whose immune system has not been stimulated by a target exogenously. In another aspect, the present invention is directed to an antibody that specifically binds to the target, wherein the antibody is identified by the above method.

In still another aspect, the present invention is directed to an antibody library for identifying an antibody to a target, which antibody library is obtained from a subject or mammal and said antibody library comprises less than 10⁷ different kinds of antibodies. In some embodiments, the antibody library is obtained from a subject or mammal whose immune system has not been stimulated by a target exogenously.

In yet another aspect, the present invention is directed to a polypeptide library, which polypeptide library comprises a plurality of isolated polypeptides comprising different, random amino acid sequences, wherein the polypeptides comprise about 5-100 amino acids, preferably, 10-20, 10-30, 10-40, 10-50, 10-60, 10-70, 10-80, 10-90, or 10-100, amino acids, the polypeptides do not comprise Cys, do not comprise 3 or more identical, consecutive amino acids, and/or do not comprise 5 or more identical amino acids. In yet another aspect, the present invention is directed to a method for producing an antibody library, which method comprises: a) immunizing a subject with the above polypeptide library; and b) recovering antibodies from said subject. In yet another aspect, the present invention is directed to an antibody library that is produced by the above method.

In yet another aspect, the present invention is directed to an isolated polypeptide set forth in the sequence listing (SEQ ID: 1-55471). In some embodiments, the present invention is directed to at least 2, 3, 4, 5, 6, 7, 8, or 9 isolated polypeptides set forth in the sequence listing (SEQ ID: 1-55471).

In yet another aspect, the present invention is directed to a polypeptide library, which polypeptide library comprises at least 10, 100, 1,000, 10,000, 15,000, 20,000, 25,000, 30,000, 35,000, 40,000, 45,000, 50,000, or all isolated polypeptides set forth in the sequence listing (SEQ ID: 1-55471). In yet another aspect, the present invention is directed to a method for producing an antibody library, which method comprises a) immunizing a subject with the above polypeptide library; and b) recovering antibodies from said subject. In yet another aspect, the present invention is directed to an antibody library that is produced by the above method.

In yet another aspect, the present invention is directed to an isolated antibody that specifically binds to a polypeptide set forth in the sequence listing (SEQ ID: 1-55471). In some embodiments, the present invention is directed to isolated antibodies that specifically bind to at least 2, 3, 4, 5, 6, 7, 8, or 9 isolated polypeptides set forth in the sequence listing (SEQ ID: 1-55471).

In yet another aspect, the present invention is directed to an antibody library, which antibody library comprises antibodies that specifically bind to at least 10, 100, 1,000, 10,000, 15,000, 20,000, 25,000, 30,000, 35,000, 40,000, 45,000, 50,000, or all polypeptides set forth in the sequence listing (SEQ ID: 1-55471).

In yet another aspect, the present invention is directed to a method for identifying a peptidic antigenic sequence to a target antibody, which method comprises: a) contacting a target antibody with a polypeptide library comprising at least 10, 100, 1,000, 10,000, 15,000, 20,000, 25,000, 30,000, 35,000, 40,000, 45,000, 50,000, or all isolated polypeptides set forth in the sequence listing (SEQ ID: 1-55471) under conditions suitable for binding between said target antibody with a polypeptide in said polypeptide library, if such polypeptide existing in said polypeptide library; and c) assessing binding between said target antibody and said polypeptide to identify a peptidic antigenic sequence to said target antibody.

In yet another aspect, the present invention is directed to an isolated antibody that specifically binds to Akt, which isolated antibody specifically binds to an epitope comprised in the amino acid sequence QDGGQKAVKD.

In yet another aspect, the present invention is directed to an isolated antibody that specifically binds to ERK2, which isolated antibody specifically binds to an epitope comprised in the amino acid sequence HPLGSPGSAS.

In yet another aspect, the present invention is directed to an isolated antibody that specifically binds to Desmin, which isolated antibody specifically binds to an epitope comprised in the amino acid sequence REIRRYQKST.

In yet another aspect, the present invention is directed to an isolated antibody that specifically binds to CBL4, which isolated antibody specifically binds to an epitope comprised in the amino acid sequence RSRARKQAYT.

In yet another aspect, the present invention is directed to an isolated antibody that specifically binds to cholera toxin, which isolated antibody specifically binds to an epitope comprised in the amino acid sequence FEEREQANTA, EYQQAQLEAE or DSSMSMADSE.

In yet another aspect, the present invention is directed to an isolated antibody that specifically binds to VEGF, which isolated antibody specifically binds to an epitope comprised in the amino acid sequence VLDFILSMGL, AKRKAGTSPR or RNSDFSAGSP.

In yet another aspect, the present invention is directed to a method for identifying a target associated with a condition, which method comprises: a) contacting a sample obtained from a source that has a condition with an antibody library, and assessing binding, or a lack thereof, between a substance in said sample and an antibody in said antibody library, wherein said antibody library is obtained from a subject or mammal whose immune system has not been stimulated by a target exogenously and said antibody library comprises less than 10⁷ different kinds of antibodies, or said antibody library is obtained by immunizing a subject or mammal with a polypeptide library, said polypeptide library comprising a plurality of isolated polypeptides comprising different, random amino acid sequences, wherein the polypeptides comprise about 5-100 amino acids, preferably, 10-20, 10-30, 10-40, 10-50, 10-60, 10-70, 10-80, 10-90, or 10-100 amino acids, the polypeptides do not comprise Cys, do not comprise 3 or more identical, consecutive amino acids, and/or do not comprise 5 or more identical amino acids, or said antibody library is obtained by immunizing a subject or mammal with a polypeptide library, said polypeptide library comprising at least 10, 100, 1,000, 10,000, 15,000, 20,000, 25,000, 30,000, 35,000, 40,000, 45,000, 50,000, or all isolated polypeptides set forth in the sequence listing (SEQ ID: 1-55471); b) contacting a sample obtained from a source that does not have said condition with the above antibody library, and assessing binding, or a lack thereof, between a substance in said sample and an antibody in said antibody library; and c) identifying a substance as a target associated with said condition when there is a difference in said binding, or a lack thereof, between said substance and said antibody in steps a) and b).

In yet another aspect, the present invention is directed to a method for identifying a target associated with a condition, which method comprises: a) contacting a sample obtained from a source that has a condition with a polypeptide library, said polypeptide library comprising a plurality of isolated polypeptides comprising different, random amino acid sequences, wherein the polypeptides comprise about 5-100 amino acids, preferably, 10-20, 10-30, 10-40, 10-50, 10-60, 10-70, 10-80, 10-90, or 10-100 amino acids, the polypeptides do not comprise Cys, do not comprise 3 or more identical, consecutive amino acids, and/or do not comprise 5 or more identical amino acids, or a polypeptide library comprising at least 10, 100, 1,000, 10,000, 15,000, 20,000, 25,000, 30,000, 35,000, 40,000, 45,000, 50,000, or all isolated polypeptides set forth in the sequence listing (SEQ ID: 1-55471), and assessing binding, or a lack thereof, between a substance in said sample and a polypeptide in said polypeptide library; b) contacting a sample obtained from a source that does not have said condition with the above polypeptide library, and assessing binding, or a lack thereof, between a substance in said sample and a polypeptide in said polypeptide library; and c) identifying a substance as a target associated with said condition when there is a difference in said binding, or a lack thereof, between said substance and said polypeptide in steps a) and b).

In yet another aspect, the present invention provides an antibody library, comprising: (1) antibodies against random peptides with 10-20 amino acids, (2) IgG antibodies, secreted by hybridoma cells produced from spleen cells of naïve mammal, (3) IgG antibodies, secreted by hybridoma cells produced from spleen cells of mammal that are immunized by total protein extract, said protein extract is from a complete organism, one or more tissues thereof, and/or one or more cells thereof, (4) IgG antibodies, secreted by stable hybridoma stains established against one or more antigens; or any combination of (1)-(4).

In one embodiment, said antibody library comprises at least 10,000 different members, and said antibody library has a success rate of at least 85% when used for screening antibodies against a target or protein of interest.

In one embodiment, the antibody library according to the present invention is in the form of hybridoma cell library.

In one embodiment, the random peptides of the invention: 1) do not contain cysteine, 2) do not contain 3 or more consecutive same amino acids, 3) do not contain 5 or more same amino acids.

In one embodiment, the initial score of each random peptide is set as any value, and the random peptides are selected through the following process: 1) for amino acids with potential glycosylation site, each potential glycosylation site reduces one point from the score, 2) each amino acid K or R reduces 4 points from the score; based on the above score, desired amount of peptides with highest score are selected from the top to the bottom.

In one embodiment, e.g., 10,000 peptides with highest score are selected from the top. In another embodiment, e.g., 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 peptides with highest score are selected from the top. In one embodiment, the selected random peptides are chemically synthesized.

In one embodiment, naïve mammal is selected from mouse, rat, and rabbit.

In one embodiment, the complete organism is selected from Arabidopsis thaliana, mouse, rat, rabbit, cattle, caprine, Drosophila, zebrafish, threadworm, rice, or maize etc.

In one embodiment, the tissues are selected from blood tissue, Arabidopsis thaliana calyx, threadworm tissues in different developmental stages, or mouse brain tissue.

In one embodiment, said one or more cells are selected from spleen cells, tumor cells or cell lines, such as human tumor cell lines.

In one embodiment, the antibodies in the antibody library have been subjected to affinity maturation. In a specific embodiment, the antibody library of the invention can be used to obtain antibodies with high affinity based on relatively small amount of library members.

In yet another aspect, the invention provides a combination, comprising an antibody library of the invention.

In another aspect, the invention provides a biochip, comprising an antibody library of the invention.

In another aspect, the invention provides a method for screening antibody against a protein of interest, comprising using the antibody library of the invention, the combination of the invention, or the biochip of the invention to screen one or more antibodies against said protein of interest.

In one embodiment, the method of the invention comprises: (a) mixing said protein of interest with antibodies or antibody groups of said antibody library, (b) selecting antibodies or antibody groups capable of binding said protein of interest.

In one embodiment, the method of the invention comprises: (a) mixing said protein of interest with antibodies or antibody groups of said antibody library, (b) selecting antibodies or antibody groups capable of binding said protein of interest, (c) mixing said protein of interest with antibodies or antibody subgroups of the antibody groups selected in step (b), (d) selecting antibodies or antibody subgroups capable of binding said protein of interest. In another embodiment, the method of the invention further comprises using the antibody subgroups selected in step (d) to repeat steps (c) and (d) until an antibody capable of binding said protein of interest is selected.

In another embodiment, the method of the invention comprises simultaneous screening against several proteins of interest, comprising: (a) mixing said several proteins of interest with antibodies or antibody groups of said antibody library, (b) selecting antibodies or antibody groups capable of binding said several proteins of interest, (c) mixing each of said several proteins of interest, separately, with the antibodies or antibody groups capable of binding said several proteins of interest selected in step (b), and then respectively selecting antibodies or antibody groups capable of binding each of said several proteins of interest.

In another embodiment, the method of the invention comprises simultaneous screening against several proteins of interest, comprising: (a) mixing said several proteins of interest with antibodies or antibody groups of said antibody library, (b) selecting antibodies or antibody groups capable of binding said several proteins of interest, (c) mixing each of said several proteins of interest, separately, with the antibodies or antibody groups capable of binding said several proteins of interest selected in step (b), and then respectively selecting antibodies or antibody groups capable of binding each of said several proteins of interest, (d) mixing each of said several proteins of interest, separately, with antibodies or antibody subgroups of the antibody groups selected in step (c) capable of binging the respective protein of interest, (e) respectively selecting antibodies or antibody subgroups capable of binding each of said several proteins of interest. In another embodiment, the method of the invention further comprises using the antibody subgroups selected in step (e) to repeat steps (d) and (e) until antibodies capable of binding each said protein of interest are respectively selected.

In one embodiment, the method of the invention can be conducted using a high-throughput screening device.

In one embodiment, the high-throughput screening device used in the method of the invention is a biochip, such as a protein chip, or a lab-on-a-chip (LOC).

In another aspect, the present invention also relates to use of the antibody library of the invention in the preparation of a device or a kit for screening antibodies against a protein of interest. In one embodiment, the device is a high-throughput screening device. In another embodiment, the high-throughput screening device is a biochip, such as a protein chip, or a lab-on-a-chip (LOC).

In one embodiment, the protein of interest is a post-translational modified protein or polypeptide, or toxic protein or polypeptide.

### BRIEF DESCRIPTION OF THE DRAWINGS

FIG. 1 illustrates an assay for phosphorylation at SER473 in AKT protein.

FIG. 2 illustrates ERK2 protein Western blotting verification.

FIG. 3 illustrates VEGF protein Western blotting verification.

FIG. 4 illustrates CBL4 protein Western blotting verification.

FIG. 5 illustrates Plasmid pHG.

FIG. 6 illustrates Plasmid pHLDis-VL.

### DETAILED DESCRIPTION OF THE INVENTION

For clarity of disclosure, and not by way of limitation, the detailed description of the invention is divided into the subsections that follow.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

The terms “polypeptide”, “oligopeptide”, “peptide” and “protein” are used interchangeably herein to refer to polymers of amino acids of any length, e.g., at least 5, 6, 7, 8, 9, 10, 20, 30, 40, 50, 100, 200, 300, 400, 500, 1,000 or more amino acids. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule, and can be an immunoglobulin of any class, e.g., IgG, IgM, IgA, IgD and IgE. IgY, which is the major antibody type in avian species such as chicken, is also included within the definition. As used herein, the term encompasses not only intact polyclonal or monoclonal antibodies, but also fragments thereof (such as Fab, Fab′, F(ab′)2, Fv), single chain (ScFv), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

As used herein, the term “specific binding” refers to the specificity of an antibody such that it preferentially binds to a target antigen, such as a polypeptide antigen. Recognition by an antibody of a particular target in the presence of other potential interfering substances is one characteristic of such binding. Preferably, antibodies or antibody fragments that are specific for or bind specifically to a target antigen bind to the target antigen with higher affinity than binding to other non-target substances. Also preferably, antibodies or antibody fragments that are specific for or bind specifically to a target antigen avoid binding to a significant percentage of non-target substances, e.g., non-target substances present in a testing sample. In some embodiments, antibodies or antibody fragments of the present disclosure avoid binding greater than about 90% of non-target substances, although higher percentages are clearly contemplated and preferred. For example, antibodies or antibody fragments of the present disclosure avoid binding about 91%, about 92%, about 93%, about 94%, about 95%, about 96%, about 97%, about 98%, about 99%, and about 99% or more of non-target substances. In other embodiments, antibodies or antibody fragments of the present disclosure avoid binding greater than about 10%, 20%, 30%, 40%, 50%, 60%, or 70%, or greater than about 75%, or greater than about 80%, or greater than about 85% of non-target substances.

As used herein, the term “specific binding” also refers to the specificity of a polypeptide such that it preferentially binds to a target antibody, such as a target antibody in a testing sample. Recognition by a polypeptide of a particular target antibody in the presence of other antibodies or substances is one characteristic of such binding. Preferably, a polypeptide that is specific for or binds specifically to an antibody binds to the target antibody with higher affinity than binding to other non-target antibodies or substances. Also preferably, a polypeptide that is specific for or binds specifically to a target antibody avoids binding to a significant percentage of non-target antibodies or substances, e.g., non-target antibodies present in a testing sample. In some embodiments, polypeptides of the present disclosure avoid binding greater than about 90% of non-target antibodies or substances, although higher percentages are clearly contemplated and preferred. For example, polypeptides of the present disclosure avoid binding about 91%, about 92%, about 93%, about 94%, about 95%, about 96%, about 97%, about 98%, about 99%, and about 99% or more of non-target antibodies or substances. In other embodiments, polypeptides of the present disclosure avoid binding greater than about 10%, 20%, 30%, 40%, 50%, 60%, or 70%, or greater than about 75%, or greater than about 80%, or greater than about 85% of non-target antibodies or substances.

As used herein, the term “antigen” refers to a target molecule that is specifically bound by an antibody through its antigen recognition site. The antigen may be monovalent or polyvalent, i.e., it may have one or more epitopes recognized by one or more antibodies. Examples of kinds of antigens that can be recognized by antibodies include polypeptides, oligosaccharides, glycoproteins, polynucleotides, lipids, etc.

The terms “polynucleotide,” “oligonucleotide,” “nucleic acid” and “nucleic acid molecule” are used interchangeably herein to refer to a polymeric form of nucleotides of any length, e.g., at least 8, 9, 10, 20, 30, 40, 50, 100, 200, 300, 400, 500, 1,000 or more nucleotides, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing nonnucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

As used herein, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. Also included are soil and water samples and other environmental samples, viruses, bacteria, fungi, algae, protozoa and components thereof.

As used herein the term “assessing” is intended to include quantitative and qualitative determination in the sense of obtaining an absolute value for the amount or concentration of the analyte present in the sample, and also of obtaining an index, ratio, percentage, visual or other value indicative of the level of analyte in the sample. Assessment may be direct or indirect and the chemical species actually detected need not of course be the analyte itself but may for example be a derivative thereof or some further substance.

As used herein, “serum” refers to the fluid portion of the blood obtained after removal of the fibrin clot and blood cells, distinguished from the plasma in circulating blood.

As used herein, “plasma” refers to the fluid, noncellular portion of the blood, distinguished from the serum obtained after coagulation.

As used herein, “production by recombinant means” refers to production methods that use recombinant nucleic acid methods that rely on well known methods of molecular biology for expressing proteins encoded by cloned nucleic acids.

As used herein, “fluid” refers to any composition that can flow. Fluids thus encompass compositions that are in the form of semi-solids, pastes, solutions, aqueous mixtures, gels, lotions, creams and other such compositions.

As used herein, “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, “disease or disorder” refers to a pathological condition in an organism resulting from, e.g., infection or genetic defect, and characterized by identifiable symptoms.

As used herein, “chip” refers to a solid substrate with a plurality of one-, two- or three-dimensional micro structures or micro-scale structures on which certain processes, such as physical, chemical, biological, biophysical or biochemical processes, etc., can be carried out. The micro structures or micro-scale structures such as, channels and wells, electrode elements, electromagnetic elements, are incorporated into, fabricated on or otherwise attached to the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips of the present invention can vary considerably, e.g., from about 1 mm² to about 0.25 m². Preferably, the size of the chips is from about 4 mm² to about 25 cm² with a characteristic dimension from about 1 mm to about 5 cm. The chip surfaces may be flat, or not flat. The chips with non-flat surfaces may include channels or wells fabricated on the surfaces.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### B. METHODS FOR IDENTIFYING AN ANTIBODY TO A TARGET

In one aspect, the present invention is directed to a method for identifying an antibody to a target, which method comprises: a) providing for an antibody library obtained from a subject, e.g., a mammal, wherein said antibody library comprises less than 10⁷ different kinds of antibodies; b) contacting said target with said antibody library under conditions suitable for binding between said target with an antibody in said antibody library, if such antibody existing in said antibody library; and c) assessing binding between said target and said antibody to identify said antibody as an antibody to said target.

Any suitable antibody library can be used in the present methods. In some embodiments, the antibody library is obtained from a subject or mammal whose immune system has not been stimulated by a target exogenously. Typically, the subject or mammal has not been immunized, or at least not actively immunized, with the target in an isolated to purified form. In some examples, the subject or mammal has not been immunized, or at least not actively immunized, with a composition that contains the target. In other examples, the subject or mammal has not been immunized, or at least not actively immunized, with a composition wherein the target constitutes a significant portion or percentage thereof. For instances, if the target is a particular polypeptide, the subject or mammal may have been immunized with a cell, tissue or organism that contains the target polypeptide. However, because the cell, tissue or organism does not contain a significant amount of the target polypeptide, or target polypeptide does not constitute a significant portion or percentage of the cell, tissue or organism, the subject or mammal is still considered as not having stimulated by the target polypeptide exogenously.

In some embodiments, the amino acid sequences of antibodies in the antibody library are unknown priori. For example, the antibodies in the antibody library can be obtained from a host through proper immunization, and the amino acid sequences of antibodies remain unknown. In other examples, the antibodies in the antibody library can be obtained initially from a host through proper immunization. Once the antibodies are obtained, the amino acid sequences of antibodies can be determined by any suitable methods, e.g., protein sequencing. In this case, the amino acid sequences of antibodies in the antibody library are unknown priori because the antibodies are not synthesized de novo but are produced from the immunization with target libraries. In contrast, Mao et al., Nature, 28(11):1195-1178 (2010) describes de novo synthesis of an antibody library and the amino acid sequences of antibodies in its antibody library are known priori.

In some embodiments, the antibodies in the antibody library comprise intact (or complete) antibody molecules. For example, if the antibodies in the antibody library are obtained from a mammal, the antibodies in the antibody library can comprise intact (or complete) structure of IgG, IgM, IgA, IgD and/or IgE molecules. If the antibodies in the antibody library are obtained from an avian species such as chicken, the antibodies in the antibody library can comprise intact (or complete) structure of IgY molecules.

The antibody library can be produced by any suitable methods using any suitable immunogens. In some embodiments, the antibody library is produced by a mammal immunized with a plurality of polypeptides comprising different, random amino acid sequences. The polypeptides can comprise any suitable number of amino acids. In some embodiments, the polypeptides comprise about 5-100 amino acids, preferably, 10-20, 10-30, 10-40, 10-50, 10-60, 10-70, 10-80, 10-90, or 10-100, amino acids. The polypeptides can comprise any suitable types and/or sequences of amino acids. In some embodiments, the polypeptides do not comprise Cys, do not comprise 3 or more identical, consecutive amino acids, and/or do not comprise 5 or more identical amino acids.

The polypeptides used to produce the antibody library can be selected by any suitable standards or methods. In some embodiments, the polypeptides are selected by the standards: a) assigning an initial, identical score to all candidate polypeptides; b) reducing the initial score by 1 point for each potential glycosylation site in a candidate polypeptide; c) reducing the initial score by 4 points for each Lys or Arg residue in a candidate polypeptide; and d) selecting candidate polypeptide with highest possible scores for the desired number of polypeptides for immunizing the mammal.

Any suitable numbers of the polypeptides can be used to immunize mammals to produce the antibody library. In some embodiments, at least 10,000 polypeptides are used to immunize mammals to produce the antibody library. In some embodiments, at least 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different polypeptides are used to immunize mammals to produce the antibody library. Typically, a single mammal is immunized with smaller number of the polypeptides and multiple mammals are immunized to cover the intended numbers of immunization. In some embodiments, a single mammal can be immunized with about 1, 2, 3, 4, 5, 10, 15, 20 25 or 25 different polypeptides. For example, if a single mammal is immunized with 10 different polypeptides and 100,000 different polypeptides are intended to be used, 10,000 mammals can be immunized to cover the intended use of the 100,000 different polypeptides.

The polypeptides can comprise any suitable number of amino acids. In some embodiments, the polypeptides can comprise 5-100 amino acids, preferably, 10-20, 10-30, 10-40, 10-50, 10-60, 10-70, 10-80, 10-90, or 10-100, amino acids. In some embodiments, the polypeptides can comprise about 10, 11, 12, 13, 14, 15, 16, 17, 18, 19 or 20 amino acids. The polypeptides can comprise any suitable types of amino acids. In some embodiments, the polypeptides can comprise natural and/or non-natural amino acids. The polypeptides can be produced by any suitable methods. In some embodiments, the polypeptides are produced by chemical synthesis and/or recombinant production.

In some embodiments, the polypeptides are selected from candidate polypeptides by the following standard: a) each of the candidate polypeptides comprises about 10 amino acids that do not include Cys; b) each of the candidate polypeptides does not comprise 3 or more identical, consecutive amino acids, c) each of the candidate polypeptides does not comprise 5 or more identical amino acids; d) each of the candidate polypeptides is assigned an initial score of 10; e) reducing the initial score by 1 point for each potential glycosylation site in a candidate polypeptide; f) reducing the initial score by 4 points for each Lys or Arg residue in a candidate polypeptide; and g) selecting at least 10,000 candidate polypeptides with the highest scores.

Any suitable subject or mammal can be used to produce the antibody library. In some embodiments, the antibody library is produced by a subject or mammal whose immune system has not been purposely stimulated to produce antibody to the intended target. In some embodiments, the mammal is a mouse, a rat, a rabbit, a goat, a bovine species such as an ox, cow, or buffalo, a canine species such as a dog, a porcine or swine species such as a pig, or a horse. In some embodiments, the mammal can be a human. Other non-mammal subjects, e.g., an avian species such as a chicken (Gallus), can also be used to produce the antibody library. Other exemplary avian species includes a quail (Coturnix), a turkey (Meleagris gallopavo), a duck, a goose and a Japanese quail (Coturnix japonica).

In some embodiments, the antibody library can be produced by immunizing a subject or mammal with an intact organism, an tissue, an cell or a whole protein extract of the organism, tissue or cell, wherein the intact organism, tissue, cell is immunologically distinct from the target. For example, the target may not be, or may not be expected to be, comprised in the intact organism, tissue, cell or a whole protein extract of the organism, tissue or cell, used as the immuogens. In another example, the target may be comprised in the intact organism, tissue, cell or a whole protein extract of the organism, tissue or cell used as the immuogens, but only constitute a small or insignificant portion of the intact organism, tissue, cell or a whole protein extract of the organism, tissue or cell.

Any suitable intact organism can be used. In some embodiments, an Arabidopsis thaliana, a mouse, a rat, a rabbit, a bovine, a goat, a Drosophila, a zebrafish, a Caenorhabditis elegans, rice or corn can be used.

Any suitable tissue can be used. In some embodiments, blood, Arabidopsis thaliana bud, Caenorhabditis elegans tissues at different developmental stages, or a mouse brain tissue can be used.

Any suitable cell can be used. In some embodiments, a spleen cell, a tumor cell or a cell line, e.g., a human tumor cell line, can be used.

In some embodiments, the antibody library is produced by a mammal immunized with an antigen that is immunologically distinct from the target.

In some embodiments, the antibody library: a) is produced by a mammal immunized with a plurality of polypeptides comprising different, random amino acid sequences; b) is produced by a mammal whose immune system has not been purposely stimulated; c) is produced by a mammal immunized with an intact organism, an tissue, an cell or a whole protein extract of the organism, tissue or cell; d) is produced by a mammal immunized with an antigen that is immunologically distinct from the target; or e) a combination of any of a)-d).

The antibody library can comprises any suitable types of antibodies. For example, the antibody library can comprise polyclonal antibodies, monoclonal antibodies and/or hybridomas that produce monoclonal antibodies. The antibodies in the antibody library can be isolated, purified, treated and/or modified after they obtained from the subject or mammal.

In some embodiments, the antibodies in the antibody library are affinity matured. In immunology, affinity maturation is the process by which B cells produce antibodies with increased affinity for antigen during the course of an immune response. With repeated exposures to the same antigen, a host will produce antibodies of successively greater affinities. A secondary response can elicit antibodies with several log fold greater affinity than in a primary response. Like the natural prototype, the in vitro affinity maturation is based on the principles of mutation and selection. The in vitro affinity maturation has successfully been used to optimize antibodies, antibody fragments or other peptide molecules like antibody mimetics. Random mutations inside the CDRs can be introduced using any suitable methods, e.g., radiation, chemical mutagens or error-prone PCR. In addition, the genetical diversity can be increased by chain shuffling. Two or three rounds of mutation and selection using display methods like phage display often results in antibodies with affinities in the low nanomolar range. See e.g., Roskos et al., (2007). Stefan Dübel. ed. Handbook of Therapeutic Antibodies. Weinheim: Wiley-VCH. pp. 145-169.

The antibody library can comprise any suitable number of different antibodies. In some embodiments, the antibody library comprises at least 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different antibodies.

The antibodies in the antibody library can be stored, transported and/or used in any suitable form for format. In some embodiments, at least some of the antibodies are immobilized on a solid surface. In some embodiments, at least 1%, 5%, 10%, 20%, 30%, 40%, 50%, 60%, 70%, 80%, 90%, 95%, or all of the antibodies are immobilized on a solid surface. Any suitable solid surface can be used. For example, the solid surface can be a part of a tube, e.g., a test tube, a well on a plate, e.g., a microtiter plate, a bead, or a biochip.

The antibody library can be screened in any suitable manner or format. For example, hybridoma cells can be obtained and antibody genes can be cloned from the hybridoma cells. Antibody molecules can be expressed from the encoding genes. Antibody molecules can be placed in a suitable assay format, e.g., an ELISA plate, for the screening. In another example, ascites or purified antibodies can be used in the screening. In still another example, hybridoma cells can be directly cultured and the supernatants can be used in the screening.

The antibody library can be screened in any suitable assay format. In some embodiments, the target-antibody complex may be assessed by a format such as enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, u-capture assay, inhibition assay or avidity assay. In some embodiments, the target-antibody complex may be assessed in a homogeneous or a heterogeneous assay format.

The target can be any suitable substances. Exemplary target includes a cell, cellular organelle, a viruse, a particle, a molecule, or an aggregate or complex thereof, or an aggregate or complex of molecules. Exemplary cell can be an organic or inorganic molecule. Exemplary organic molecule can be an amino acid, a peptide, a protein, e.g., an antibody or receptor, a nucleoside, a nucleotide, an oligonucleotide, a nucleic acid, e.g., DNA or RNA, a vitamin, a monosaccharide, an oligosaccharide, a carbohydrate, a lipid, or a complex thereof. In some embodiments, the target is a polypeptide. Exemplary polypeptide includes a linear polypeptide, a soluble polypeptide, a modified polypeptide, a toxic polypeptide or a polypeptide that causes autoimmunity in a subject.

The target can be contacted with the antibodies in the antibody library in any suitable manner or order. For example, the target can be contacted with all antibodies in the antibody library at once or at the same time. Alternatively, the target can be contacted with portions of the antibodies in the antibody library, either in parallel or sequentially.

In some embodiments, the target is contacted with a subgroup of antibodies in the antibody library to determine if the subgroup of antibodies comprises an antibody that specifically binds to the target. Once it is determined that the subgroup of antibodies comprises an antibody that specifically binds to the target, the method can further comprises the steps: a) dividing the subgroup of antibodies into a smaller subgroup of antibodies; and b) contacting the target to determine if the smaller subgroup of antibodies comprises an antibody that specifically binds to the target. In some embodiments, the steps a) and b) can be repeated until an individual antibody that specifically binds to the target is identified.

The present method can be used to identify antibodies that specifically bind to any suitable number of target. In some embodiments, the present method can be used to identify antibodies that specifically bind to a single target. In some embodiments, the present method can be used to identify antibodies that specifically bind to a plurality of the targets, e.g., 2, 3, 4, 5, 6, 7, 8, 9, 10, 20, 30, 40, 50, 60, 70, 80, 90, 100 or more targets.

In some embodiments, the antibody library is contacted with the plurality of the targets to identify antibodies or groups of antibodies that specifically bind to the plurality of the targets. In some embodiments, the method can further comprise contacting each of the plurality of the targets with the identified antibodies or groups of antibodies to identify antibodies or groups of antibodies that specifically bind to each of the plurality of the targets.

In some embodiments, the method can further comprise: a) dividing the identified antibodies or groups of antibodies into smaller subgroups of antibodies; and b) contacting each of the plurality of the targets with the smaller subgroups of antibodies to determine if the smaller subgroup of antibodies comprises an antibody that specifically binds to each of the plurality of the targets. In some embodiments, the steps a) and b) can be repeated until an individual antibody that specifically binds to each of the plurality of the targets is identified.

The present method can be conducted to achieve any suitable, intended or desired successful rate for identifying an antibody that specifically binds to the intended target. Generally, the successful rate depends on one or more factors, such as the type of target, the number of the target, the size of the antibody library, the types and quality of the antibodies in the antibody library, the procedures for producing the antibodies or the antibody library, and screening assay formats, etc. In some embodiments, the successful rate for identifying an antibody that specifically binds to the target is at least 10%, 20%, 30%, 40%, 50%, 60%, 70%, 80%, 90%, 95%, 96%, 97%, 98%, 99%, or 100%. In some embodiments, the antibody library comprises at least 10,000 different antibodies and the successful rate for identifying an antibody that specifically binds to the target is at least 80%, 85%, 90%, 95%, 96%, 97%, 98%, 99%, or 100%.

Although an antibody library obtained from a mammal is used above to describe or illustrate the method for identifying an antibody to a target, an antibody library obtained from a non-mammal subject, e.g., an avian species such as a chicken, can also be used in the present methods.

In another aspect, the present invention is directed to an antibody that specifically binds to the target, wherein the antibody is identified by the above method.

### C. ANTIBODIES, ANTIBODY LIBRARIES, POLYPEPTIDE, POLYPEPTIDE LIBRARIES AND USES THEREOF

In still another aspect, the present invention is directed to an antibody library for identifying an antibody to a target, which antibody library is obtained from a subject or an mammal and said antibody library comprises less than 10⁷ different kinds of antibodies. In some embodiments, the antibody library is obtained from a subject or an mammal whose immune system has not been stimulated by a target exogenously.

The antibody library can be produced by any suitable methods. In some embodiments, the antibody library: a) is produced by a mammal immunized with a plurality of polypeptides comprising different, random amino acid sequences; b) is produced by a mammal whose immune system has not been purposely stimulated; c) is produced by a mammal immunized with an intact organism, an tissue, an cell or a whole protein extract of the organism, tissue or cell; d) is produced by a mammal immunized with an antigen that is immunologically distinct from the target; or e) a combination of any of a)-d).

The antibody library can comprise any suitable number of antibodies. In some embodiments, the antibody library comprises at least 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different antibodies.

The antibody library can comprise any suitable types of antibodies. In some embodiments, the antibody library comprises polyclonal antibodies, monoclonal antibodies and/or hybridomas that produce monoclonal antibodies. In some embodiments, the amino acid sequences of antibodies in the antibody library are unknown priori. In some embodiments, the antibodies in the antibody library comprise intact (or complete) antibody molecules.

The antibodies in the antibody library can be isolated, purified, treated and/or modified after they obtained from the subject or mammal. In some embodiments, the antibodies in the antibody library are affinity matured.

In yet another aspect, the present invention is directed to a polypeptide library, which polypeptide library comprises a plurality of isolated polypeptides comprising different, random amino acid sequences, wherein the polypeptides comprise about 5-100 amino acids, preferably, 10-20, 10-30, 10-40, 10-50, 10-60, 10-70, 10-80, 10-90, or 10-100 amino acids, the polypeptides do not comprise Cys, do not comprise 3 or more identical, consecutive amino acids, and/or do not comprise 5 or more identical amino acids. In some embodiments, the polypeptides can comprise about 10, 11, 12, 13, 14, 15, 16, 17, 18, 19 or 20 amino acids.

The polypeptides can be selected by any suitable standards or methods. In some embodiments, the polypeptides are selected by the standards: a) assigning an initial, identical score to all candidate polypeptides; b) reducing the initial score by 1 point for each potential glycosylation site in a candidate polypeptide; c) reducing the initial score by 4 points for each Lys or Arg residue in a candidate polypeptide; and d) selecting candidate polypeptide with highest possible scores for the desired number of polypeptides.

The polypeptide library can comprise any suitable number of polypeptides. In some embodiments, the polypeptide library comprises at least 10, 100, 1,000, 10,000, 15,000, 20,000, 25,000, 30,000, 35,000, 40,000, 45,000, 50,000, 60,000, 70,000, 80,000, 90,000, 100,000 or more different polypeptides. In some embodiments, the polypeptide library comprises at least 10,000 different polypeptides.

The polypeptides can comprise any suitable types of amino acids. In some embodiments, the polypeptides can comprise natural and/or non-natural amino acids. The polypeptides can be produced by any suitable methods. In some embodiments, the polypeptides are produced by chemical synthesis and/or recombinant production.

The polypeptide library can be used for any suitable purposes. For example, the polypeptide library can be used to produce an antibody library.

In yet another aspect, the present invention is directed to a method for producing an antibody library, which method comprises: a) immunizing a subject with a polypeptide library as disclosed above; and b) recovering antibodies from said subject.

The antibodies in the antibody library can be isolated, purified, treated and/or modified after they obtained from the subject or mammal. In some embodiments, the antibodies in the antibody library are affinity matured. In some embodiments, the present method can further comprise affinity purifying antibodies recovered from the subject using a polypeptide library as disclosed above.

The subject can be immunized with the polypeptides in the library in any suitable manner or order. Typically, a single subject or mammal is immunized with smaller number of the polypeptides and multiple mammals are immunized to cover the intended numbers of immunization. In some embodiments, a single subject or mammal can be immunized with about 1, 2, 3, 4, 5, 10, 15, 20 25 or 25 different polypeptides. For example, if a single mammal is immunized with 10 different polypeptides and 100,000 different polypeptides are intended to be used, 10,000 mammals can be immunized to cover the intended use of the 100,000 different polypeptides. In some embodiments, a subject or mammal is immunized with a group of about 5-20 polypeptides, e.g., 5, 6, 7, 8, 9, 10, 11, 12, 13, 14, 15, 16, 17, 18, 19 or different polypeptides, in the library, and multiple subjects are immunized with multiple groups of about 5-20 polypeptides in the library, and the multiple groups of about 5-20 polypeptides encompass all the polypeptides in the library.

In yet another aspect, the present invention is directed to an antibody library, which is produced by the present methods.

The antibody library can comprise any suitable number of antibodies. In some embodiments, the antibody library comprises at least 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different antibodies. The antibody library can comprise any suitable types of antibodies. In some embodiments, the antibody library comprises polyclonal antibodies, monoclonal antibodies and/or hybridomas that produce monoclonal antibodies. In some embodiments, the amino acid sequences of antibodies in the antibody library are unknown priori. In some embodiments, the antibodies in the antibody library comprise intact (or complete) antibody molecules.

The antibodies in the antibody library can be isolated, purified, treated and/or modified after they obtained from the subject or mammal. In some embodiments, the antibodies in the antibody library are affinity matured.

In yet another aspect, the present invention is directed to an isolated polypeptide set forth in the sequence listing (SEQ ID: 1-55471). In some embodiments, the present invention is directed to at least 2, 3, 4, 5, 6, 7, 8, or 9 isolated polypeptides set forth in the sequence listing (SEQ ID: 1-55471). The isolated polypeptide(s) can be in any suitable form of composition, combination, complex, kit or article of manufactures. The isolated polypeptide(s) can be made by any suitable methods, e.g., chemical synthesis, recombinant production or a combination thereof.

In yet another aspect, the present invention is directed to a polypeptide library, which polypeptide library comprises at least 10, 100, 1,000, 10,000, 15,000, 20,000, 25,000, 30,000, 35,000, 40,000, 45,000, 50,000, or all isolated polypeptides set forth in the sequence listing (SEQ ID: 1-55471).

The polypeptide library can be used for any suitable purposes. For example, the polypeptide library can be used to produce an antibody library.

In yet another aspect, the present invention is directed to a method for producing an antibody library, which method comprises a) immunizing a subject with the polypeptide library as disclosed above; and b) recovering antibodies from said subject.

The antibodies in the antibody library can be isolated, purified, treated and/or modified after they obtained from the subject or mammal. In some embodiments, the antibodies in the antibody library are affinity matured. In some embodiments, the present method can further comprise affinity purifying antibodies recovered from the subject using a polypeptide library as disclosed above.

In yet another aspect, the present invention is directed to an antibody library, which is produced by the method as disclosed above.

The antibody library can comprise any suitable number of antibodies. In some embodiments, the antibody library comprises at least 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different antibodies.

The antibody library can comprise any suitable types of antibodies. In some embodiments, the antibody library comprises polyclonal antibodies, monoclonal antibodies and/or hybridomas that produce monoclonal antibodies. In some embodiments, the amino acid sequences of antibodies in the antibody library are unknown priori. In some embodiments, the antibodies in the antibody library comprise intact (or complete) antibody molecules.

The antibodies in the antibody library can be isolated, purified, treated and/or modified after they obtained from the subject or mammal. In some embodiments, the antibodies in the antibody library are affinity matured.

In yet another aspect, the present invention is directed to an isolated antibody that specifically binds to a polypeptide set forth in the sequence listing (SEQ ID: 1-55471). In some embodiments, the present invention is directed to isolated antibodies that specifically bind to at least 2, 3, 4, 5, 6, 7, 8, or 9 isolated polypeptides set forth in the sequence listing (SEQ ID: 1-55471). The isolated antibody or antibodies can be in any suitable form of composition, combination, complex, kit or article of manufactures. The isolated antibody or antibodies can be made by any suitable methods, e.g., immunization of a host, various display technology, e.g., phage display technology, hybridoma technology, recombinant production or a combination thereof.

In yet another aspect, the present invention is directed to an antibody library, which antibody library comprises antibodies that specifically bind to at least 10, 100, 1,000, 10,000, 15,000, 20,000, 25,000, 30,000, 35,000, 40,000, 45,000, 50,000, or all polypeptides set forth in the sequence listing (SEQ ID: 1-55471).

The antibody library can comprise any suitable types of antibodies. In some embodiments, the antibody library comprises polyclonal antibodies, monoclonal antibodies and/or hybridomas that produce monoclonal antibodies. In some embodiments, the amino acid sequences of antibodies in the antibody library are unknown priori. In some embodiments, the antibodies in the antibody library comprise intact (or complete) antibody molecules.

The antibodies in the antibody library can be isolated, purified, treated and/or modified after they obtained from the subject or mammal. In some embodiments, the antibodies in the antibody library are affinity matured.

### D. METHODS FOR IDENTIFYING A PEPTIDIC ANTIGENIC SEQUENCE TO A TARGET ANTIBODY

In yet another aspect, the present invention is directed to a method for identifying a peptidic antigenic sequence to a target antibody, which method comprises: a) contacting a target antibody with a polypeptide library comprising at least 10, 100, 1,000, 10,000, 15,000, 20,000, 25,000, 30,000, 35,000, 40,000, 45,000, 50,000, or all isolated polypeptides set forth in the sequence listing (SEQ ID: 1-55471) under conditions suitable for binding between said target antibody with a polypeptide in said polypeptide library, if such polypeptide existing in said polypeptide library; and c) assessing binding between said target antibody and said polypeptide to identify a peptidic antigenic sequence to said target antibody.

The target antibody can be contacted with the polypeptides in the polypeptide library in any suitable manner or order. For example, the target antibody can be contacted with all polypeptides in the polypeptide library at once or at the same time. Alternatively, the target antibody can be contacted with portions of the polypeptides in the polypeptide library, either in parallel or sequentially.

In some embodiments, the target antibody is contacted with a subgroup of polypeptides in the polypeptide library to determine if the subgroup of polypeptides comprises a polypeptide that specifically binds to the target antibody. Once it is determined that the subgroup of polypeptides comprises a polypeptide that specifically binds to the target antibody, and method can further comprise: a) dividing the subgroup of polypeptides into a smaller subgroup of polypeptides; and b) contacting the target antibody with the smaller subgroup of polypeptides to determine if the smaller subgroup of polypeptides comprises a polypeptide that specifically binds to the target antibody. The steps a) and b) can be repeated until an individual polypeptide that specifically binds to the target antibody is identified.

The present methods can be used to identify a peptidic antigenic sequence to a single target antibody. The present methods can also be used to identify peptidic antigenic sequences to a plurality of target antibodies.

Once the peptidic antigenic sequence to a target antibody is identified, the present methods can comprise further post-identification steps. In some embodiments, the method can further comprise isolating the polypeptide that specifically binds to the target antibody. In some embodiments, the method can further comprise determining amino acid sequence of the isolated polypeptide.

The present methods can be used for any suitable purposes. In some embodiments, the present methods can be used for identifying a peptidic antigenic sequence to a target antibody that is a biomarker, e.g., a diagnostic or prognostic biomarker.

### E. ISOLATED ANTIBODIES TO SPECIFIC TARGETS

In yet another aspect, the present invention is directed to an isolated antibody that specifically binds to Akt, which isolated antibody specifically binds to an epitope comprised in the amino acid sequence QDGGQKAVKD.

In yet another aspect, the present invention is directed to an isolated antibody that specifically binds to ERK2, which isolated antibody specifically binds to an epitope comprised in the amino acid sequence HPLGSPGSAS.

In yet another aspect, the present invention is directed to an isolated antibody that specifically binds to Desmin, which isolated antibody specifically binds to an epitope comprised in the amino acid sequence REIRRYQKST.

In yet another aspect, the present invention is directed to an isolated antibody that specifically binds to CBL4, which isolated antibody specifically binds to an epitope comprised in the amino acid sequence RSRARKQAYT.

In yet another aspect, the present invention is directed to an isolated antibody that specifically binds to cholera toxin, which isolated antibody specifically binds to an epitope comprised in the amino acid sequence FEEREQANTA, EYQQAQLEAE or DSSMSMADSE.

In yet another aspect, the present invention is directed to an isolated antibody that specifically binds to VEGF, which isolated antibody specifically binds to an epitope comprised in the amino acid sequence VLDFILSMGL, AKRKAGTSPR or RNSDFSAGSP.

The above antibody can be any suitable types of antibodies. In some embodiments, the antibody can be polyclonal antibodies, monoclonal antibodies and/or hybridomas that produce monoclonal antibodies.

The above antibody can be produced by any suitable methods, by immunizing a host with a target polypeptide, by phage display or recombinant production, etc. The above antibody can be further purified, treated and/or modified. In some embodiments, the above antibody can be affinity matured.

### F. METHODS FOR IDENTIFYING A TARGET ASSOCIATED WITH A CONDITION

In yet another aspect, the present invention is directed to a method for identifying a target associated with a condition, which method comprises: a) contacting a sample obtained from a source that has a condition with an antibody library, and assessing binding, or a lack thereof, between a substance in said sample and an antibody in said antibody library, wherein said antibody library is obtained from a subject or mammal, and preferably whose immune system has not been stimulated by a target exogenously, and said antibody library comprises less than 10⁷ different kinds of antibodies, or said antibody library is obtained by immunizing a subject or mammal with a polypeptide library, said polypeptide library comprising a plurality of isolated polypeptides comprising different, random amino acid sequences, wherein the polypeptides comprise about 10-20 amino acids, the polypeptides do not comprise Cys, do not comprise 3 or more identical, consecutive amino acids, and/or do not comprise 5 or more identical amino acids, or said antibody library is obtained by immunizing a subject with a polypeptide library, said polypeptide library comprising at least 10, 100, 1,000, 10,000, 15,000, 20,000, 25,000, 30,000, 35,000, 40,000, 45,000, 50,000, or all isolated polypeptides set forth in the sequence listing (SEQ ID: 1-55471); b) contacting a sample obtained from a source that does not have said condition with the above antibody library, and assessing binding, or a lack thereof, between a substance in said sample and an antibody in said antibody library; and c) identifying a substance as a target associated with said condition when there is a difference in said binding, or a lack thereof, between said substance and said antibody in steps a) and b).

The present methods can be used for any suitable purposes. In some embodiments, the present method is used for identifying a target associated with a condition in a subject. In some embodiments, the present method is used for identifying a target associated with a disease or disorder.

The present methods can be used for identifying a target associated with a single condition. Alternatively, the present methods can be used for identifying multiple targets associated with a condition. Still alternatively, the present methods can be used for identifying multiple targets associated with a single condition. Yet alternatively, the present methods can be used for identifying multiple targets associated with multiple conditions.

The difference in the binding, or a lack thereof, between the substance and the antibody can be assessed in any suitable manner. In some embodiments, the difference in the binding, or a lack thereof, between the substance and the antibody in steps a) and b) is qualitative. In some embodiments, the difference in the binding, or a lack thereof, between the substance and the antibody in steps a) and b) is quantitative. In some embodiments, binding between the substance and the antibody in step a) and lack of the binding between the substance and the antibody in step b) identify the substance as a target associated with the condition. In some embodiments, lack of binding between the substance and the antibody in step a) and binding between the substance and the antibody in step b) identify the substance as a target associated with the condition.

The target can be any suitable substances. Exemplary target includes a cell, cellular organelle, a viruse, a particle, a molecule, or an aggregate or complex thereof, or an aggregate or complex of molecules. Exemplary cell can be an organic or inorganic molecule. Exemplary organic molecule can be an amino acid, a peptide, a protein, e.g., an antibody or receptor, a nucleoside, a nucleotide, an oligonucleotide, a nucleic acid, e.g., DNA or RNA, a vitamin, a monosaccharide, an oligosaccharide, a carbohydrate, a lipid, or a complex thereof. In some embodiments, the target is a polypeptide. Exemplary polypeptide includes a linear polypeptide, a soluble polypeptide, a modified polypeptide, a toxic polypeptide or a polypeptide that causes autoimmunity in a subject.

In yet another aspect, the present invention is directed to a method for identifying a target associated with a condition, which method comprises: a) contacting a sample obtained from a source that has a condition with a polypeptide library, said polypeptide library comprising a plurality of isolated polypeptides comprising different, random amino acid sequences, wherein the polypeptides comprise about 10-20 amino acids, the polypeptides do not comprise Cys, do not comprise 3 or more identical, consecutive amino acids, and/or do not comprise 5 or more identical amino acids, or a polypeptide library comprising at least 10, 100, 1,000, 10,000, 15,000, 20,000, 25,000, 30,000, 35,000, 40,000, 45,000, 50,000, or all isolated polypeptides set forth in the sequence listing (SEQ ID: 1-55471), and assessing binding, or a lack thereof, between a substance in said sample and a polypeptide in said polypeptide library; b) contacting a sample obtained from a source that does not have said condition with the above polypeptide library, and assessing binding, or a lack thereof, between a substance in said sample and a polypeptide in said polypeptide library; and c) identifying a substance as a target associated with said condition when there is a difference in said binding, or a lack thereof, between said substance and said polypeptide in steps a) and b).

The present methods can be used for any suitable purposes. In some embodiments, the present method is used for identifying a target associated with a condition in a subject. In some embodiments, the present method is used for identifying a target associated with a disease or disorder.

The present methods can be used for identifying a target associated with a single condition. Alternatively, the present methods can be used for identifying multiple targets associated with a condition. Still alternatively, the present methods can be used for identifying multiple targets associated with a single condition. Yet alternatively, the present methods can be used for identifying multiple targets associated with multiple conditions.

The difference in the binding, or a lack thereof, between the substance and the antibody can be assessed in any suitable manner. In some embodiments, the difference in the binding, or a lack thereof, between the substance and the polypeptide in steps a) and b) is qualitative. In some embodiments, the difference in the binding, or a lack thereof, between the substance and the polypeptide in steps a) and b) is quantitative. In some embodiments, binding between the substance and the polypeptide in step a) and lack of binding between the substance and the polypeptide in step b) identify the substance as a target associated with the condition. In some embodiments, lack of binding between the substance and the polypeptide in step a) and binding between the substance and the polypeptide in step b) identify the substance as a target associated with the condition.

The target can be any suitable substances. Exemplary target includes a cell, cellular organelle, a viruse, a particle, a molecule, or an aggregate or complex thereof, or an aggregate or complex of molecules. Exemplary cell can be an organic or inorganic molecule. Exemplary organic molecule can be an amino acid, a peptide, a protein, e.g., an antibody or receptor, a nucleoside, a nucleotide, an oligonucleotide, a nucleic acid, e.g., DNA or RNA, a vitamin, a monosaccharide, an oligosaccharide, a carbohydrate, a lipid, or a complex thereof. In some embodiments, the target is a polypeptide. Exemplary polypeptide includes a linear polypeptide, a soluble polypeptide, a modified polypeptide, a toxic polypeptide or a polypeptide that causes autoimmunity in a subject. In some embodiments, the target comprises an antibody.

### G. EXAMPLARY EMBODIMENTS

Unless otherwise indicated, all the technical and scientific terms used herein will have their common meanings known in the art. All the patents, patent applications, publications, GenBank sequences, websites and other disclosed material will be incorporated herein as references, unless otherwise indicated.

With respect to any antibody against a protein, it generally recognizes only a few amino acids of the protein. When the number of different antibodies prepared against random amino acid sequences is sufficient, these antibodies can constitute an antibody library. Using such library, screening can be conducted against a protein of interest, so as to obtain antibodies capable of recognizing the protein.

Furthermore, as for a particular organism, such as a mouse, if the total protein extract from a certain tissue thereof is used to immunize an animal, and then a corresponding antibody library can be prepared, this antibody library can also be used for screening antibodies with high affinity to a certain protein of the organism. Naïve mammal can be employed for hybridoma fusion, so as to screen hybridoma cells that are capable of secreting IgG antibodies, these hybridoma cells can also be used to construct an antibody library for screening antibodies.

Accordingly, In some embodiments, the invention provides an antibody library for screening antibodies, and a method for screening antibodies against a protein of interest using said antibody library. Specifically, the invention relates to an antibody library with at least 10,000 different members, which can be used for screening antibodies with high affinity to a protein of interest.

In one aspect, the present invention provides an antibody library, comprising: (1) antibodies against random peptides with 10-20 amino acids, (2) IgG antibodies, secreted by hybridoma cells produced from spleen cells of naïve mammal, (3) IgG antibodies, secreted by hybridoma cells produced from spleen cells of mammal that are immunized by total protein extract, said protein extract is from a complete organism, one or more tissues thereof, and/or one or more cells thereof, (4) IgG antibodies, secreted by stable hybridoma stains established against one or more antigens; or any combination of (1)-(4).

In some embodiments, the term “antibody library” refers to a collection of a series of antibodies, it can contain antibodies of various origins, such as antibodies produced against specific epitopes, or antibodies produced against random peptides of a protein of interest.

The antibody library of the invention can be an antibody library with antibodies of a single origin; it can also be an antibody library mixture of antibodies of various origins.

In some embodiments, the term “naïve mammal” refers to an animal that has never been stimulated or treated using experimental means. In some embodiments, it specifically refers to an animal that has never been vaccinated or immunized by foreign antigens, said animal can be: mouse, rat, rabbit etc.

In some embodiments, the term “total protein extract” refers to the collection of all the proteins originated from a complete organism, a tissue thereof, or a cell thereof. Said organism can be various model organisms, such as, Arabidopsis thaliana, mouse, mice, rabbit, cattle, caprine, Drosophila, zebrafish, threadworm, maize, or rice etc.

In some embodiments, the term “random peptide” used herein refers to randomly generated amino acid sequence, wherein said amino acid is selected from natural amino acids or analogs thereof. In the present invention, the length of a random peptide can be e.g., 10-20 amino acids, such as a random peptide of 10 amino acids.

In one embodiment, the random peptides of the invention: 1) do not contain cysteine, 2) do not contain 3 or more consecutive same amino acids, and/or 3) do not contain 5 or more same amino acids.

In one embodiment, the initial score of each random peptide is set as any value, and the random peptides are selected through the following process: 1) for amino acids with potential glycosylation site, each potential glycosylation site reduces one point from the score, 2) each amino acid K or R reduces 4 points from the score; based on the above score, desired amount of peptides with highest score are selected from the top to the bottom.

In one embodiment, the random peptides of the invention are selected through the following steps: 1) randomly generating peptide sequences with 10-20 amino acids, which do not contain cysteine, 2) the initial score for each random peptide is equal to the number of amino acids contained therein; for amino acids with potential glycosylation site, each potential glycosylation site reduces one point from the score, 3) said peptide sequences with 10-20 amino acids are not allowed to contain 3 or more consecutive same amino acids, 4) said peptide sequences with 10-20 amino acids are not allowed to contain 5 or more same amino acids, 5) each amino acid K or R in said peptide sequences with 10-20 amino acids reduces 4 points from the score. Based on the above scoring principle, peptides with highest score are selected from the top to the bottom.

In one embodiment, e.g., 10,000 peptides with highest score are selected from the top. In another embodiment, e.g. 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 peptides with highest score are selected from the top. In one embodiment, the selected random peptides are chemically synthesized.

In a specific embodiment, using random peptides with 10 amino acids as examples, the selection of random peptides can e.g. comprise the following steps: 1) Randomly generating peptide sequences with 10 amino acids, which do not contain cysteine; 2) The sorting principle of the peptide sequences: the initial score for each of the randomly generated peptide is set as 10; for amino acids with potential glycosylation site, each potential glycosylation site reduces one point from the score; 3) Said peptide sequences with 10 amino acids are not allowed to contain 3 or more consecutive same amino acids; 4) Said peptide sequences with 10 amino acids are not allowed to contain 5 or more same amino acids; and 5) Each amino acid K or R in said peptide sequences will reduce 4 points from the score.

Based on the above scoring principle, 10,000 peptides with highest score can be selected from the top to the bottom, and can then be chemically synthesized.

In an embodiment of the invention, the random peptides can be obtained by various methods, such as chemical synthesis, recombinant expression etc. Such technical means are well known in the art.

The immunization of animals can be conducted using any methods known in the art. The animal used for immunization in the present invention can be animals commonly used in the art, such as mouse, rat, rabbit, sheep, goat, horse, cattle etc.

In one embodiment, said antibody library comprises at least 10,000 different members, and said antibody library has a success rate of at least 85% when used for screening antibodies against proteins of interest. In another embodiment, the number of the members in the antibody library according to the invention can be further increased with the addition of new antibodies, such as at least 15,000, 20,000, 25,000, 30,000, 35,000, 40,000, 45,000, 50,000, 55,000, 60,000, 65,000, 70,000, 75,000, 80,000, 85,000, 90,000, 95,000, 100,000 different antibodies, and even more. With the increase of the amount of antibodies in the antibody library, the success rate of the antibody library for screening antibodies against proteins of interest will increase accordingly, such as 86%, 87%, 88%, 89%, 90%, 91%, 92%, 93%, 94%, 95%, 96%, 97%, 98%, 99% and even high. With the increase of the library content, the success rate can approach 100%.

In one embodiment, the antibody library according to the present invention is in the form of hybridoma cell library.

Detailed introduction of hybridoma techniques can be found in, e.g., Bazin, Rat hybridomas and rat monoclonal antibodies, CRC Press, 1990; Goding, Monoclonal antibodies: principles and practice, 3^{rd} edition, Academic Press, 1996; Shepherd and Dean Monoclonal Antibodies, Oxford University Press, 2000 etc.

The antibody library of the invention produced using random peptides can contain various collections of antibodies, such as a collection of monoclonal antibodies, and a collection of polyclonal antibodies. In an embodiment, the antibody library of the invention contains antibodies against all the proteins of a species. In another embodiment, the antibody library of the invention contains antibodies against all the epitopes of one or more proteins of interest.

In one embodiment, the antibody library of the invention exists in a high-throughput screening device.

In one embodiment, the high-throughput screening device used in the invention is biochip, such as protein chip, or lab-on-a-chip (LOC).

In some embodiments, the term “high-throughput screening device” refers to a device that can be used to conduct High Throughput Screening (HTS). High Throughput Screening refers to using experimental means of molecular level or of cellular level as basis, conducting automatic operation to perform the experimental processes on experimental carriers in the form of micro-plate, and using detection instruments to collect experimental data, then using computer to analyze and process the experimental data. High-throughput screening devices and techniques can be used to simultaneously detect a great amount of different samples, and they can be combined with the antibody library of the invention so as to achieve the purpose of screening antibodies rapidly and effectively.

Commonly used high-throughput screening devices include biochip. Biochip is a microarray technique, and can be used for high-throughput screening of biological samples. Regarding biochip, with the help of micro-processing and microelectronic techniques, a great amount of nucleic acid or protein fragments with known sequences can be orderly arranged onto the surface of micro-slides. The corresponding components or activities of the sample to be tested can be analyzed through reactions with labeled nucleic acid or protein molecules. Biochip typically can be divided into three different types, i.e., gene chip, protein chip, and lab-on-a-chip (LOC).

Protein chip is a high-throughput technique for analyzing protein functions, which can be used for analyzing the expression profile of proteins, for studying the interactions between proteins, and for studying interactions between DNA and proteins as well as RNA and proteins.

Lab-on-a-chip is a micro-analyzing system using chip as the platform, which can integrate basic operation units like the preparation and/or screening of samples, and the separation and/or detection of products onto a biochip, so as to accomplish different biological or chemical reaction processes, and thereby analyze the products. Using lab-on-a-chip, the screening, detection and/or separation of antibodies in the present invention can be rapidly and effectively performed on one ship. Detailed descriptions about Lab-on-a-chip can be found in, e.g. Herold, K E; Rasooly, A (eds): Lab-on-a-Chip Technology: Biomolecular Separation and Analysi, Caister Academic Press (2009), and Edwin Oosterbroek & A. van den Berg (eds.): Lab-on-a-Chip: Miniaturized systems for (bio)chemical analysis and synthesis, Elsevier Science, second edition (2003) etc.

In one embodiment, the antibodies in the antibody library of the invention have been subjected to affinity maturation. In a specific embodiment, the antibody library of the invention can be used to obtain antibodies with high affinity based on relatively small amount of library members.

The meaning of the term “affinity maturation” is well known in the art, and can be found in, e.g., Dong, Zhiwei et al.: Antibody Engineering, Beijing Medical University Publications (2002). Typically, the term “affinity maturation” means that, after immunizing an animal by a particular antigen, the antibodies thus produced and separated are structurally rearranged and reconstituted, so that the affinity of the protein against the particular antigen can be increased, e.g., by 3-4 orders of magnitude. In some embodiments, the antibodies subjected to affinity maturation are all antibodies of IgG subtypes. Therefore, in the case the antibodies of the antibody library are subjected to affinity maturation, the possibility of screening an antibody with high affinity from this antibody library will be greatly increased.

In another aspect, the invention provides a combination, comprising an antibody library of the invention.

The antibody library of the invention can be in the form of a combination, e.g., the antibody members of an antibody library can be prepared as antibody solutions with certain concentrations (the preparation methods include ascites and in vitro culture etc.). The prepared antibody solutions can be stored in the form of ELISA plates (e.g. 96- or 384-well plate), or in the form of chips.

Alternatively, genes encoding the antibody members in an antibody library can also be cloned from the cell strain, and the genes can then be used to prepare the antibodies.

In another aspect, the invention provides a biochip, comprising an antibody library of the invention.

In another aspect, the invention provides a method for screening antibody against a protein of interest, comprising using the antibody library of the invention, the combination of the invention, or the biochip of the invention to screen one or more antibodies against said protein of interest.

In some embodiments, the terms “a protein of interest” or “a polypeptide of interest” or “a peptide of interest” can be interchangeably used herein, and they all refer to any natural protein or fragment thereof, or an isoform of a natural protein obtained through alternative splicing, or a mutant of a natural protein, or any combination of the above proteins.

In some embodiments, the “alternative splicing” used herein refers to the process of producing different mRNA splicing isoforms from a same mRNA precursor through different splicing modes (i.e. combining exons through different splicing sites). The protein products obtained through alternative splicing are isoforms to each other, they can exhibit different functions and structural properties, or they can lead to different phenotypes due to their different expression levels in same cells.

In one embodiment, the method of the invention comprises: (a) mixing said protein of interest with antibodies or antibody groups of said antibody library, and (b) selecting antibodies or antibody groups capable of binding said protein of interest.

In one embodiment, the method of the invention comprises: (a) mixing said protein of interest with antibodies or antibody groups of said antibody library, (b) selecting antibodies or antibody groups capable of binding said protein of interest, (c) mixing said protein of interest with antibodies or antibody subgroups of the antibody groups selected in step (b), and (d) selecting antibodies or antibody subgroups capable of binding said protein of interest. In another embodiment, the method of the invention further comprises using the antibody subgroups selected in step (d) to repeat steps (c) and (d) until an antibody capable of binding said protein of interest is selected.

In another embodiment, the method of the invention comprises simultaneous screening against several proteins of interest, comprising: (a) mixing said several proteins of interest with antibodies or antibody groups of said antibody library, (b) selecting antibodies or antibody groups capable of binding said several proteins of interest, and (c) mixing each of said several proteins of interest, separately, with the antibodies or antibody groups capable of binding said several proteins of interest selected in step (b), and then respectively selecting antibodies or antibody groups capable of binding each of said several proteins of interest.

In another embodiment, the method of the invention comprises simultaneous screening against several proteins of interest, comprising: (a) mixing said several proteins of interest with antibodies or antibody groups of said antibody library, (b) selecting antibodies or antibody groups capable of binding said several proteins of interest, (c) mixing each of said several proteins of interest, separately, with the antibodies or antibody groups capable of binding said several proteins of interest selected in step (b), and then respectively selecting antibodies or antibody groups capable of binding each of said several proteins of interest, (d) mixing each of said several proteins of interest, separately, with antibodies or antibody subgroups of the antibody groups selected in step (c) capable of binging the respective protein of interest, and (e) respectively selecting antibodies or antibody subgroups capable of binding each of said several proteins of interest. In another embodiment, the method of the invention further comprises using the antibody subgroups selected in step (e) to repeat steps (d) and (e) until antibodies capable of binding each said protein of interest are respectively selected.

In some embodiments, the term “antibody group” refers to the mixture of different antibodies, it can contain several, several tens of, several hundreds of, or several thousands of different antibodies. An antibody group can be further divided into several antibody sub-group containing different antibodies.

In a specific embodiment, a person skilled in the art can divide the antibody library into several antibody groups according to particular requirements, such as groups containing several, several tens of, several hundreds of, or several thousands of different antibodies. As for instance, an antibody library containing 10,000 antibodies can be divided into 100 groups, each group contains 100 different antibodies. The protein of interest is separately mixed with each of the groups, and then the groups that can bind the protein of interest are selected. Such groups can be used for further screening. For instance, the above selected antibody group containing 100 antibodies can be further divided into 10 sub-groups, each sub-group contains 10 antibodies. The protein of interest is separately mixed with each of the sub-groups, and then the sub-groups that can bind the protein of interest are selected. According to such strategy, the screening can be repeatedly conducted until antibodies that can bind the protein of interest are selected.

In one embodiment, the antibodies can be directly used or can be used after dilution, and preferably they are used in a same concentration. For example, different antibodies can be diluted to 100 μg/ml, and equal volume of the antibody solutions can be taken can then mixed, so as to obtain a group containing different antibodies. In this way, several tens of thousands of antibodies can be prepared as a combination of antibody groups. A person skilled in the art can select suitable dilution liquids for the antibodies, such as HEPES solution, e.g., a HEPES solution containing 2 mg/ml Proclin300, 1% BSA, pH7.4.

The screening methods used in the invention can be ELISA, Dotblot or protein chips as well as other detection methods that can demonstrate the interactions between a protein and an antibody. These methods are all technical means known in the art.

In one embodiment, the method of the invention can be used for screening antibodies against linear polypeptides.

The term “linear polypeptide” refers to a consecutive amino acid sequence in a protein.

In one embodiment, the method of the invention can be used for screening antibodies against soluble polypeptides.

In one embodiment, the method of the invention can be used for screening antibodies against modified polypeptides.

The term “modified polypeptide”, “modified protein”, and “modified peptide” are interchangeably used herein, they all refer to a protein or polypeptides that has been modified or post-translationally modified, such as proteins or polypeptides that have been phosphorylated, methylated, or acetylated.

The antibody library of the invention can be used to produce different antibodies respectively against modified polypeptides and unmodified precursor polypeptides (also referred to as distinguishing polypeptide, i.e., the form of the polypeptide that has not been post-translationally modified). A cell strain that is positive to the polypeptide can be used to test the titer of the modified polypeptide and the unmodified polypeptide, respectively. When the difference between these two titers reaches a certain extent, such as larger than 8 [units?], then the antibody is considered as being capable of distinguishing these two polypeptides.

In one embodiment, the method of the invention can be used fro screening antibodies against toxic polypeptides. The term “toxic polypeptide” and “toxic protein” is used interchangeably herein, and they both refer to a protein or a polypeptide that can produce toxicity in an animal or in a cell. Due to its toxicity, conventional antibody preparation method cannot be used to produce antibodies with high affinity to the toxic polypeptide

In one embodiment, the method of the invention can be conducted using a high-throughput screening device. In one embodiment, the high-throughput screening device used in the method of the invention is a biochip, such as protein chip, or lab-on-a-chip (LOC).

In another aspect, the present invention also relates to use of the antibody library of the invention in the preparation of device or kit for screening antibodies against a protein of interest. In one embodiment, the device is high-throughput screening device. In another embodiment, the high-throughput screening device is a biochip, such as protein chip, or lab-on-a-chip (LOC).

Using the antibody library of the invention, antibodies against different types of proteins (linear, soluble, unmodified, modified) can be obtained. More than 50% of the antibodies obtained using the antibody library of the invention have an affinity lower than 100 nM. And the possibility of obtaining an applicable cell strain is between several in ten thousands and one in several tens of thousands, which is much higher than conventional display techniques. As for conventional phage display library, even though the content of the library reaches the level of 10⁶, the success rate of screening an applicable antibody therefrom is almost 0, this is because the affinity of the obtained antibodies normally cannot fulfill the requirements for applications.

The antibody library of the invention is based on the principle of relative specificity, said antibody library contains antibodies against tens of thousands of antigens, and thereby monoclonal antibodies with high affinity against the target antigen can be obtained in a short time (e.g., one week). The time period and cost thereof are much lower than conventional monoclonal techniques. For normal protein antigens, the affinity of thus obtained antibody shows no substantive difference when compared to antibodies obtained in conventional methods. Furthermore, the content of the antibody library according to the present invention is continuously increasing, and with the increase in the content of the library, the success rate of screening antibodies will rapidly increase accordingly.

Additionally, the screening method of the invention can also be used for screening antibodies against antigens whose antibodies cannot be prepared using conventional methods, such as toxic, or autoimmune antigens.

The present invention accomplishes the technical method for preparing antibodies with high affinity in a low-cost and high-throughput way, which can be used to prepare antibodies in a short time. And the antibody library as well as the screening method of the invention can also be combined with high-throughput techniques like protein chip.

### D. EXAMPLES

The present invention will be further illustrated in detail through the following examples. These examples are provided merely for illustrative purpose, and they should not be considered as limitation for the scope of the invention. Specifically, the present invention contains the following examples:

Example 1 describes the construction of an antibody library containing 10000 antibodies, and verifies that the success rate of screening antibodies against 20 different proteins is 85%;

Example 2 describes the construction of an antibody library containing 50000 antibodies;

Example 3 describes the screening of antibodies against modified peptides;

Example 4 describes the screening and detection of antibodies against ERK2 protein;

Example 5 describes the screening and detection of antibodies against soluble protein Desmin;

Example 6 describes the screening of antibodies against toxic protein cholera toxin, and the affinity maturation of the obtained antibodies;

Example 7 describes the screening of antibody against insoluble protein; and

Example 8 describes the preparation of antibody biochip, and the screening of antibodies against human vascular endothelial growth factor (VEGF) using said biochip.

### Example 1

### The Construction of an Antibody Library

This example describes the construction of an exemplary antibody library.

### A. Producing Antibodies Using Random Peptides

The generation of random peptides:
i. Randomly generating peptide sequences with 10 amino acids, which do not contain cysteine;
ii. The sorting principle of the peptide sequences: the initial score for each of the randomly generated peptide was set as 10; for amino acids with potential glycosylation site, each potential glycosylation site reduced one point from the score;
iii. Said peptide sequences with 10 amino acids did not contain 3 or more consecutive same amino acids;
iv. Said peptide sequences with 10 amino acids did not contain 5 or more same amino acids;
v. Each amino acid K or R in said peptide sequences reduced 4 points from the score; and
vi. Based on the above score, 10,000 peptides with highest score were selected from the top to the bottom, and were then chemically synthesized (the synthetic methods can be found in Chemistry of Peptide Synthesis, N. Leo Benoiton, 2005).

The preparation of peptide antigens, the immunization method and the preparation of monoclonal antibodies are all common techniques known in the art, descriptions about these techniques can be found in relevant publications and textbooks, e.g., Bazin, Rat hybridomas and rat monoclonal antibodies, CRC Press, 1990; Goding, Monoclonal antibodies: principles and practice, 3^{rd} edition, Academic Press, 1996; Shepherd and Dean Monoclonal antibodies, Oxford University Press, 2000 etc.

B. Producing Antibodies Using Spleen Cells of Naïve Mice

The method for preparing monoclonal antibodies can be found in e.g., Bazin, Rat hybridomas and rat monoclonal antibodies, CRC Press, 1990; Goding, Monoclonal antibodies: principles and practice, 3^{rd} edition, Academic Press, 1996; Shepherd and Dean Monoclonal antibodies, Oxford University Press, 2000 etc.

Lymphocytes were taken from the spleen of a mouse that had not been subjected to immunization, and hybridoma cells were then prepared through cell fusion; caprine-anti-mouse IgG antibody (Abmart, 20100815) was used to detect hybridoma cells that can secret antibodies.

Specifically: the caprine-anti-mouse IgG antibody was diluted to 1 μg/ml using 0.01M Na₂CO₃/NaHCO₃ buffer (pH 9.0), and was then added into 96-well ELISA plate with high adsorption capacity (SYBIO, Hangzhou, China), 100 μl was added into each well, coating at 4° C. overnight, washing with PBST for 3 times, 250 μl/well washing solution was added each time. 250 μl blocking solution (PBST solution containing 1% BSA) was added into each well, blocking at 37° C. for 1 h, washing with PBST 3 times, 250 μl/well washing solution was added each time. 20 μl supernatant was taken from each well of the cell fusion plate, supplementing 80 μl blocking solution, incubating at 37° C. for 1 h, removing the remaining solution in the plate, washing with PBST 3 times, 250 μl/well washing solution was added each time. 100 μl HRP-labeled caprine-anti-mouse antibody (Abmart, 20110228) was added into each well, incubating at 37° C. for 1 h, washing with PBST for 5 times, 250 μl/well washing solution was added each time. Solution of horseradish peroxidase substrate TMB (Sigma) was added, incubating at 37° C. for 15 min, 50 μl 2M H₂SO₄ solution was added into each well to stop the reaction, the absorption value was read at 450 nm.

C. Producing Antibodies by Immunizing Animals with Total Protein Extract

Protein preparation: HeLa cells was lysed using RIPA buffer (50 mM Tris pH7.4, 150 mM NaCl, 1% Triton-X-100, 1% sodium deoxycholate 0.1% SDS lysis solution) containing protease inhibitor (Roche), and was quantitated by BCA (Biocolors, Shanghai, China).

The preparation of peptide antigens, the immunization method and the preparation of monoclonal antibodies are all common techniques known in the art, descriptions about these techniques can be found in relevant publications and textbooks, e.g., Bazin, Rat hybridomas and rat monoclonal antibodies, CRC Press, 1990; Goding, Monoclonal antibodies: principles and practice, 3^{rd} edition, Academic Press, 1996; Shepherd and Dean Monoclonal antibodies, Oxford University Press, 2000 etc.

### D. Constructing Antibody Library

The purified antibodies obtained in the above steps A, B, and C were taken and mixed, so as to constitute an antibody library. Each 100 different antibodies of the antibody library were mixed to form antibody sub-libraries, and altogether 100 sub-libraries were obtained.

### E. Screening Antibodies Against Proteins Using the Constructed Antibody Library Containing 10,000 Antibodies

Twenty (20) soluble proteins with 100-600 amino acids in length were used as target proteins, so as to verify the success rate of the antibody library containing 10,000 antibodies for screening antibodies against proteins with special conformation.

Said target proteins were all purchased from Shanghai PrimeGene Bio-Tech LTD, the specific proteins can be seen in Table 2 below.

TABLE 2
Screening specific antibodies for recombinant proteins

| | | The number of the |
|---|---|---|
| | | obtained positive |
| Protein No. | Protein Name | stains |
| 1 | Recombinant Human OSM | 3 |
| 2 | Recombinant Human Angiostatin | 1 |
| 3 | Recombinant Human BCMA | 2 |
| 4 | Recombinant Human EMAP-II | 2 |
| 5 | Recombinant Human IL-2 | 2 |
| 6 | Recombinant Human FGF-basic | 0 |
| 7 | Recombinant Human KGF1 | 1 |
| 8 | Recombinant Human FGF-9 | 1 |
| 9 | Recombinant Human IL-7 | 1 |
| 10 | Recombinant Human IL-6 | 2 |
| 11 | Recombinant Human IL-10 | 3 |
| 12 | Recombinant Human EGF | 4 |
| 13 | Recombinant Human IL-12 | 2 |
| 14 | Recombinant Human IL-13 | 1 |
| 15 | Recombinant Human EPG | 0 |
| 16 | Recombinant Human IL-15 | 1 |
| 17 | Recombinant Human IL-17 | 1 |
| 18 | Recombinant Human IFN-α1b | 0 |
| 19 | Recombinant Human IL-20 | 2 |
| 20 | Recombinant Human IL-21 | 3 |

The 20 proteins were formulated as antigens into solutions with the concentration thereof at 0.2 ug/ml, and they were separately used to coat 100 ELISA plates, 100 μl of said solutions were added into each well, coating at 4° C. overnight. ELISA method was adopted to screen positive antibody combinations (the specific method can be seen in step B of Example 1). The antibodies in the antibody library were diluted at 1:16,000 (0.02M PH7.4 phosphate buffer), and were then used to detect whether they can recognize the antigens. An ELISA OD value over 1.0 was defined as positive. The obtained antibody sub-library that can recognize a single protein was considered as an antibody sub-library of interest.

As for the antibody sub-library that can recognize a single protein, the respective protein was used as antigen to separately detect the 100 antibodies in the sub-library, so as to obtain positive cell strains that can recognize the respective protein. The specific screening results can be seen in Table 2. For the 20 proteins, the success rate of obtaining at least 1 specific antibody was 85%.

Additionally, with the increasing in the content of the library, the success rate for screening was further increased.

### Example 2

### The Construction of an Antibody Library with 50,000 Antibodies

The content of the antibody library was further increased. The new antibody library contained all the antibodies of the antibody library constructed in Example 1, antibodies derived from non-immunized mice and from mice immunized by total protein extracts, as well as monoclonal antibodies obtained by immunizing mice with more peptides. 15,000 peptides (the sequences of which are set forth in SEQ ID: 1-15,000) were used to immunize mice so as to prepare antibodies, 3 strains with highest titer were selected for each peptide, those peptides (3,000) that could not be successfully used to prepare antibodies were excluded, 36,000 strains were successfully obtained using the other 12,000 peptides (SEQ ID: 1-12,000). All the above monoclonal antibodies were collected to construct an antibody library containing 50,000 antibodies. The origins of the different antibodies are shown in Tables 3 and 4. According to the antigen design principle, altogether 54,771 peptides (SEQ ID: 1-55471) with 10 amino acids were collected in Abmart peptide library.

The antibody library containing 50,000 antibodies was divided into 500 antibody groups, each group contained 100 different antibodies.

TABLE 3
The origins of the different antibodies in
the antibody library with 50,000 members

| The origin of antibodies | The number of antibodies |
|---|---|
| Non-immniyed mice | 1,000 |
| The mice immunized by total protein extracts | 3,000 |
| Immunizing mice with peptides with 10 a.a. | 36,000 |
| Anidodies from Example 1 | 10,000 |

TABLE 4
Total protein extracts

| The origin of the antigens | Protein types |
|---|---|
| Huamn serum | Toal proteins(excluding seralbumin) |
| | glycoprotein |
| Arabidopsis thaliana | calyx |
| | immature stage lamina |
| | immature stage root |
| rice, maize | lamina |
| | root |
| | anther |
| mouse, rat, rabbit, cattle, caprine | brain tissue |
| | serum total proteins |
| | liver |
| | kidney |
| Drosophila, zebrafish, | total protein extacts |
| threadworm | |
| Cell lines | 293T |
| | HELA |
| | human cholangiocarcinoma cell RBE |
| | huamn breast cancer cell strain MCF-7 |
| | HEPG2 |

Based on antibody libraries with different contents, the success rates for different projects are summarized in Table 5. The increase of the library content could significantly increase the success rates for the screening against various antigens. Using the antibody library with 50,000 members as basis, 3,000-5,000 new monoclonal antibodies were added into the library each much (based on the 54,771 peptides). The higher level of the library content can further increase the success rate.

TABLE 5
The effects of different library contents on the success rate

| | The number of screening | Library content | | |
|---|---|---|---|---|
| Project types | projects | 1,000 | 10,000 | 50,000 |
| Modified | 100 | 0 | 10 | 43 |
| polypeptides | | | | |
| Soluble proteins | 100 | 26 | 87 | 98 |
| Insoluble proteins | 100 | 12 | 50 | 90 |

The antibody library used in the following Examples 3-8 was the antibody library with about 50,000 antibodies constructed in Example 2.

### Example 3

### Screening Antibodies Against Modified Peptides

This example describes the screening of antibodies against modified peptides. The obtained antibodies can distinguish the modified peptides from the respective unmodified ones.

The modified peptides and the respective unmodified peptides were synthesized by Scilight-Peptide Inc., Beijing, China, and the purities of all the synthesized peptides were more than 85%.

The peptide sequences were designated as: p-protein name-modification site.

TABLE 6
The modified peptide sequences

| | Sequence of the | Sequence of the |
|---|---|---|
| Name of the peptides | modified peptides | unmodified peptides |
| p-Smad2/3(Ser423/425) | PSIRCS(pS)V(pS) | PSIRCSSVS |
| p-Stat3 | C-SAAP(pY)LKTKFI | C-SAAPYLKTKFI |
| p-Stat1(Tyr701) | C-KGTG(pY)IKTELI | C-KGTGYIKTELI |
| p-Akt (Thr308) | ATMK(pT)FCGT | ATMKIFCGT |
| p-Akt (Ser473) | C-HFPQF(pS)YSAS | C-HFPQFSYSAS |
| p-JNK(Thr 183/Tyr 185) | C-SFMM(pT)P(pY)VVTR | C-SFMMTPYVVTR |
| p-ERK1/2(Thr202/Tyr204) | HTGFL(pT)E(pY)VAC | HTGFLTEYVAC |
| p-IRS1(Tyr989) | C-SRGD(pY)MTMQM | C-SRGDYMTMQM |
| p-IRS1(Tyr632) | C-GSGD(pY)MPMSP | C-GSGDYMPMSP |
| p-IRS1(Ser307) | C-SRTE(pS)ITATS | C-SRTESITATS |
| p-IRS1(Tyr941) | C-TGTEE(pY)MKMDL | C-TGTEEYMKMDL |
| P-C-Jun(C-J4C4/1) | C-HITT(pT)P(pT)PTQ | C-HITTTP(pT)PTQ |
| p-Cdk5(Ser159) | RCY(pS)AEVVTLW | RCYSAEVVTLW |
| p-Cdk5(Tyr15) | C-GEGT(pY)GTVFK | C-GEGTYGTVFK |
| p-EGFR(Tyr1172) | C-DNPD(pY)QQDF | C-DNPDYQQDF |
| p-EGFR(Ser1045) | C-ATSNN(pS)TVA | C-ATSNNSTVA |

### A. The Coupling of Peptide Antigens

The peptides as antigens were coupling with BAS through glutaral method, detailed description of the method can be seen in, e.g., The Protein Protocols Handbook, Cytogen, Princeton, Humana press.

### B. Screening the Peptide-Specific Antibodies

The screening method was similar to step E in Example 1.

Specifically: first, the 16 modified peptides were used as antigens, to separately screen positive antibody sub-libraries that recognize the respective antigen. According to the screening results, the screened positive antibody sub-libraries were used as the basis to further screen antibodies against a single modified peptide.

Based on the screened positive wells, the coupled modified peptides and coupled unmodified peptides were separately used as antigens to detect whether the screened antibodies can distinguish these two kinds of peptides. In the case the antibody to be tested can bind a modified peptide and show an OD value larger than 3 times of the corresponding OD value of the respective unmodified peptide, then the antibody was considered as capable of distinguishing these two kinds of peptides, i.e., the antibody can specifically recognize modified peptide.

The detailed screening results can be seen in Table 7.

TABLE 7
The screening results for modified peptides

| | The num- | The Number | The Number of |
|---|---|---|---|
| | ber of | of antibodies | antibodies that |
| | positive | that recog- | specifically |
| | sub- | nize modi- | recognize modi- |
| Protein | libraries | fied peptides | fied peptides |
| p-Smad2/3(Ser423/425) | 5 | 8 | 1 |
| p-Stat3 | 5 | 4 | 0 |
| p-Stat1(Tyr701) | 0 | 0 | 0 |
| p-Akt (Thr308) | 9 | 13 | 2 |
| p-Akt (Ser473) | 10 | 12 | 2 |
| p-JNK(Thr 183/Tyr 185) | 0 | 0 | 0 |
| p-ERK1/2(Thr202/Tyr204) | 0 | 0 | 0 |
| p-IRS1(Tyr989) | 6 | 8 | 1 |
| p-IRS1(Tyr632) | 7 | 7 | 2 |
| p-IRS1(Ser307) | 0 | 0 | 0 |
| p-IRS1(Tyr941) | 0 | 0 | 0 |
| P-C-Jun(C-J4C4/1) | 0 | 0 | 0 |
| p-Cdk5(Ser159) | 7 | 8 | 1 |
| p-Cdk5(Tyr15) | 0 | 0 | 0 |
| p-EGFR(Tyr1172) | 0 | 0 | 0 |
| p-EGFR(Ser1045) | 0 | 0 | 0 |

### C. The Screened Positive Antibodies can Recognize Phosphorylated Akt Protein

Western blotting process was as following: Insulin-treated and untreated 293T cells (ATCC, CRL-11268™) were lysed with RIPA buffer (50 mM Tris pH7.4, 150 mM NaCl, 1% Triton-X-100, 1% sodium deoxycholate 0.1% SDS lysis solution) containing protease inhibitor (Roche), and was quantitated by BCA (Biocolors, Shanghai, China), diluting with 5× loading buffer, after denaturing at 100° C. for 10 min, 20-30 ng sample was loaded in each lane, and 10% SDS-PAGE was used for gel electrophoresis, blocking with 5% skim milk after PVDF membrane transfer, the primary antibody was abmart-anti-Akt (Phospho-Ser473), after diluting the respective ascites of the mice at 1:500, incubating at room temperature for 1 h, washing with 1×PBST for 3 times, 5 min for each time; the secondary antibody was abmart-anti-mouse-HRP, after diluting with PBST solution of 5% skim milk at 1:5000, putting into incubation box, incubating at room temperature for 30 min, washing with 1×PBST for 3×5 min, ECL Plus (Amersham) was used for detection.

Western detection results showed that, the screened antibodies can specifically recognize phosphorylated Akt protein in the insulin induced 293T cells (see FIG. 1).

### Example 4

### Screening Antibodies Against ERK2 Protein

This example describes the screening and detection of antibodies against ERK2 protein.

### A. Screening Antibodies Against ERK2 Protein

ERK2 protein was purchased form Sino Biological Inc., Beijing, China. The method for screening antibodies can be seen in the above examples. Altogether 5 strains of positive antibodies were screened from the antibody library, wherein two of them had an affinity lower than 10 nM.

### B. Western Blotting Process

The cell line used for Western blotting detection was Hela cell line (ATCC, CCL-2.2™), the treating process was identical to Example 3.

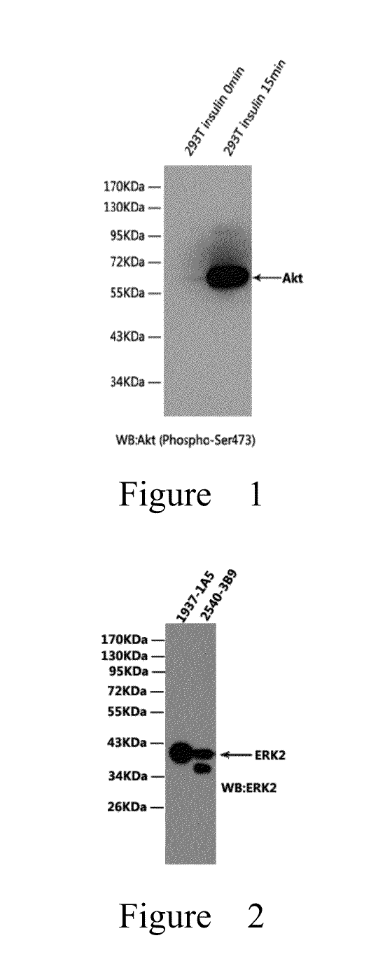

Western detection results showed that, the screened antibodies can specifically recognize the ERK2 protein in Hela cells. Antibody 1937-1A5 (Abmart, 20100605) was a monoclonal antibody that had been proved to specifically recognize ERK2 protein (commercialized antibody for detecting ERK2); while antibody 2540-3B9 was a specific antibody screened from the antibody library (see FIG. 2), which can also recognize ERK2 protein.

### Example 5

### Screening Specific Antibody Against Soluble Protein Desmin

This example describes the screening and detection of antibodies against soluble protein Desmin.

### A. Screening Antibodies Against Desmin Protein

Desmin protein was purchased from ProSpec (USA). The method for screening antibodies can be seen in the above examples. Altogether 6 strains of positive antibodies were screened from the antibody library, wherein 1 of them had an affinity lower than 10 nM.

### B. Immunohistochemistry Verification of Desmin Protein

Desmin protein is specifically highly expressed protein in cervical cancer tissue, therefore cervical cancer tissue section was used to verify the specificity and efficacy of antibodies against Desmin protein. Cervical cancer tissue section was purchased from Fengfan Medical Science Development LTD., Luohe, China, the method concerning the section and the detection can be seen in, e.g. (Immunohistochemistry Experimental Techniques and Applications, 2006, Chemical Industry Publication, Beijing, China). The antibodies obtained from the screening in the antibody library were used to detect the tissue section, the clone number of said antibody was 1956-1NB-2E7. Said antibody (ascites) was diluted at 1:500 with PBST solution containing 5% skim milk), the secondary antibody was caprine-anti-mouse-labeled HRP (Abmart, 1:5000).

The tissue section was fixed and then placed on optical microscope for observation. The IHC results showed that, antibody 1956-1NB-2E7 can specifically detect Desmin protein in the tissue, i.e., it had good tissue-specificity.

### Example 6

### The Screening of Cholera Toxin-Specific Antibodies, and Affinity Maturation Thereof

This example describes the screening of antibodies against toxic protein cholera toxin, and the affinity maturation of the obtained antibodies.

As for bacterial toxins like cholera toxin and kreotoxin, due to their high toxicity, conventional method of immunizing mouse cannot be used to prepare specific antibodies against them. In order to demonstrate the distinct advantages of the antibody library for screening antibodies against such proteins, cholera toxin was selected as antigen for screening antibodies against it.

Cholera toxin was purchased from MACGENE TECH., Beijing, China. The method for screening antibodies was identical to step E in Example 1. Altogether 3 strains of positive antibodies were screened from the antibody library, wherein 1 of them had an affinity lower than 10 nM. In competitive ELISA process, this antibody can have a detection sensitivity of 10 ng/ml for standard.

Affinity maturation of cholera toxin-specific antibodies: The method for perdorming affinity maturation is light chain shuffling method, principle of this method can be seen in ANTIBODY ENGINEERING, Methods in Molecular Biology, 2004, Volume 248, III, 327-343.

1. The obtaining of mouse variable region of light chain (VL) and variable region of heavy chain (VH). The amplification method: Rohatgi S, Ganju P, Sehgal D. Systematic design and testing of nested (RT-)PCR primers for specific amplification of mouse rearranged/expressed immunoglobulin variable region genes from small number of B cells. J Immunol Methods. 2008; 339(2):205-1. The obtained variable regions can be seen in Table 8 below.

TABLE 8
The obtained variable regions

| 1948-1- | |
|---|---|
| 1-3B5 | Sequnce |
| VH | GVQLQQSGAELVKPGASVKLSCTASGFKIKDTYMHWMKQR |
| | PEQGLEWIGRIDPANGNSHYDPKIQGKATMTADKSSNTAY |
| | LQLSSLTSEDTAVYYCASIYYGNNYVMDYWGQGTSVTVSS |
| VK | DVLMTQTPLSLPVSLGDQASISCRSSQTMVHSNGNTYLEW |
| | YLQKPGQSPKLLIYKVSNRFSGVPDRFSGSGSGTDFTLKI |
| | SRVEAEDLGVYYCFQGSLVPLTFGAGTKVELK |

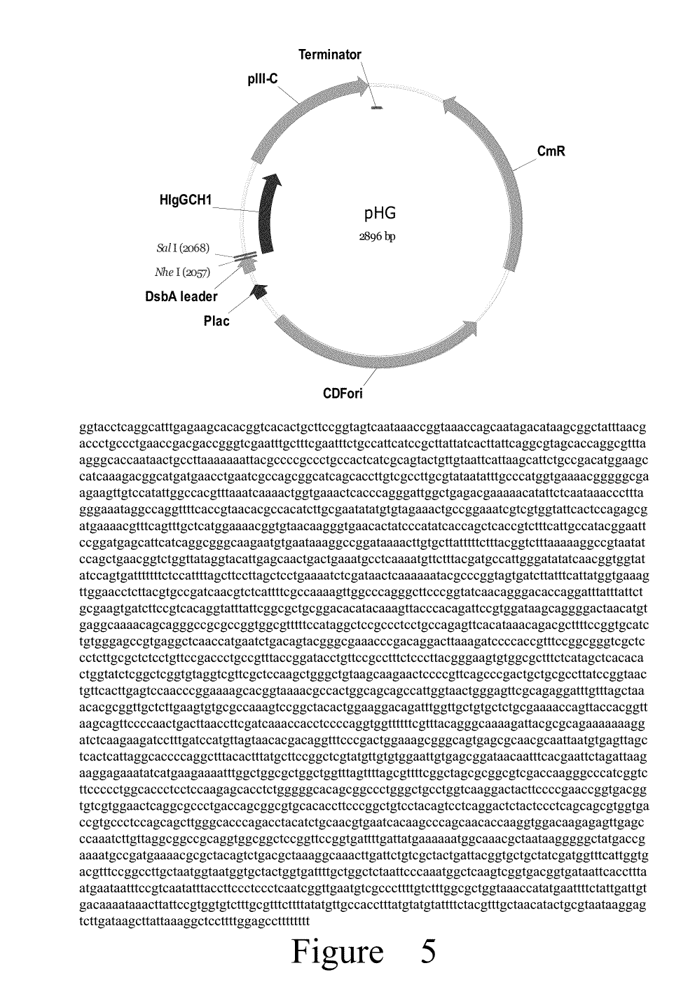

2. The VH genes were cloned through SalI and NheI restriction sites into the pHG vector (Abmart, see FIG. 5) of the double vector display system, the vector contained constant region of heavy chain CH1, which can be used to display Fab heavy chain antibody.

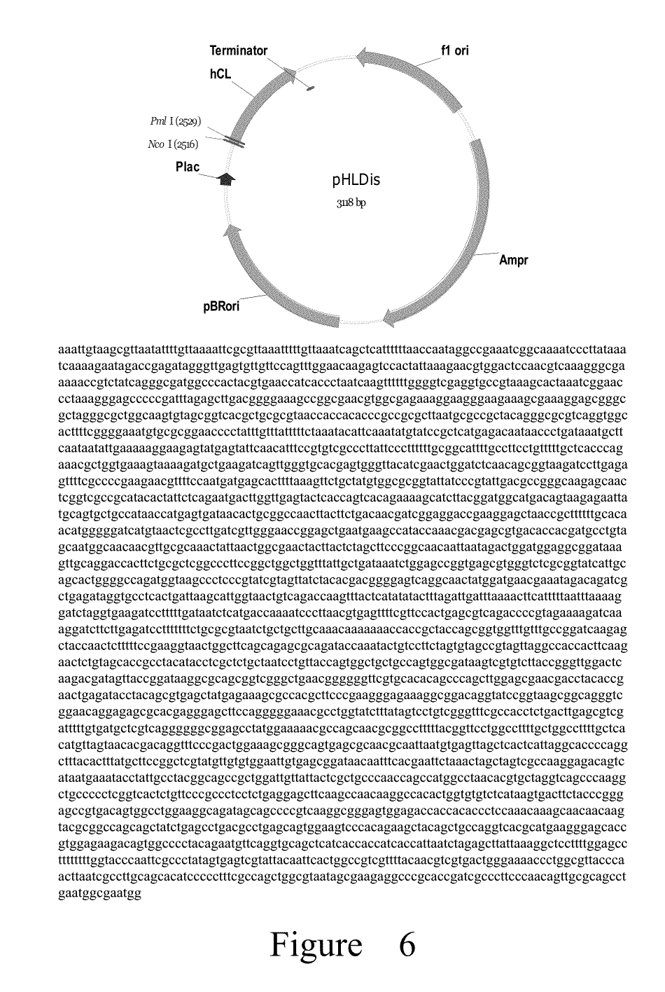

3. The pHG plasmid containing the VH gene and the constructed humanized light chain antibody library pHLDis-VL (Abmart, de novo synthesized, see FIG. 6) were mixed at a ratio of 1:1, and the mixture was then electronically transformed into TransMax competent cells (Takara, Dalian, China), all the transformed strains were collected and formed an affinity-matured antibody library.

4. Standard method was used to prepare affinity-matured phage antibody library, and to prepare phage antibody displaying library for screening (the method can be seen in Amersham biosciences: Expression Module/Recombinant Phage Antibody System).

5. The screened phage library was used to infect TransMax strain containing pHG plasmid, performing the next round of screening, monoclones were picked for detection after 2-3 rounds of screeening, phage ELISA was used to detect and identify the sensitivity of the antibodies (the method can be seen in Amersham biosciences: Expression Module/Recombinant Phage Antibody System).

6. After detecting the sensitivity of the phage antibodies against cholera toxin, the detection limit of the monoclonal 1948-3B5-1C12 against the target reached 1.2 ng/ml.

7. After sequencing, the sequence of the Kappa chain in the obtained antibody with high affinity is shown in Table 9, with the amino acids in bold letters (and with underline) as the differences to the original sequence.

TABLE 9

| 1948-1-1- | |
|---|---|
| 3B5-1C13 | Sequence |
| Vκ-orig- | DVLMTQTPLSLPVSLGDQASISCRSSQTMVHSNGNTYL |
| inal cell | EWYLQKPGQSPKLLIYKVSNRFSGVPDRFSGSGSGTDF |
| strain | TLKISRVEAEDLGVYYCFQGSLVPLTFGAGTKVELK |
| Vκ-new | DVLMTQTPLSLPVSLGDQASISCRSSQSIVHSNGNTYL |
| cell | EWYLQKPGQSPKLLIYKVSNRFSGVPDRFSGSGSGTDF |
| strain | TLKISRVEAEDLGFYYCFQGSHLPLTFGAGTKLELK |

### Example 7

### Screening Specific Antibody Against Insoluble Protein

This example describes the screening of antibody against insoluble protein.

CBL4 protein (24 KD) of maize family, which have 211 amino acids in its full length format, was obtained through recombinant expression in E. coli. expression system (Abmart, 20100504). This protein can resolve in denaturation solution of 8M urea, but is insoluble in non-denaturation solution.

The method for screening antibodies was essentially identical to step E in Example 1, except that, PH 7.0 8M urea solution was used as coating solution for insoluble proteins, and the protein concentration of the coating solution was 0.2 ug/ml.

Altogether 5 strains of positive antibodies were screened from the antibody library, wherein one of them succeeded in a verification cell line in which the protein was over-expressed (the target protein was expressed with GFP as fusion protein-50 KD). Western blotting experiments were conducted in two modes, i.e., against said positive antibody (1233-6G5) and against GFP (Abmart, 1:1000) respectively. The bands showed in the two modes are identical and both of them are at the correct size, demonstrating that the antibody library at 50,000 level can be used for screening antibodies against insoluble proteins (see FIG. 4).

### Example 8

### The Preparation of Antibody Biochip and the Screening of Protein Specific Antibodies

This example describes the preparation of antibody biochip, and the screening of antibodies against human vascular endothelial growth factor (VEGF) using said biochip.

Preparing the antibody biochip: the antibody library sample was loaded in aliquots into 384-well cell culture plate, CapitalBio SmartArrayer™ 48 spotter and CapitalBio 3 dimensional H-group slides (CapitalBio, Beijing China) were used; blocking the chip: 30 ml PBS solution containing 10 mg/ml BSA was used to block the chip by shaking at room temperature for 1 h; washing: TBST solution (30 ml each time) was used to wash the chip twice, the interval between each time was 5 min; the chip was taken out and the water remaining on the surface was removed, the chip was kept as not completely dry, and was stored at −80° C.

Human vascular endothelial growth factor (VEGF) was purchased from PeproTech, the sample was dialyzed using PBS, and was then concentrated using ultrafiltration tube (10 K), the concentration of the protein was measured (determined as over 1 mg/ml); biotin labeling: Pierce NHS activated biotin was used, the required amount of the biotin was calculated (the biotin was kept as powder, and was freshly formulated before use). The sample was kept at room temperature for 1 h, and 1M Tris (pH 7.2, the molar ratio between Tris and biotin was 5:1) was then added to stop the reaction. Desalting column was used to conduct the desalinization for 4-5 times, the sample was then divided into aliquots and stored frozen.

Directly dropping 1 ml biotin-labeled antigens (<2 μg/ml, TBST solution containing 10 mg/ml BSA) or adding 100 μl with the help of cover slip; standing at room temperature for 1 h; washing with TBST solution for 5 times, shaking thoroughly during each washing; taking out the chip and removing the water remaining on the surface, and the chip was kept as not completely dry. Directly dropping 1 ml fluorescein-labeled streptavidin (1000 times diluted, TBST solution of 10 mg/ml), standing at room temperature for 1 h; washing with TBST solution for 4 times, 5 min each time, shaking thoroughly, then rinsing with distilled water for 3 times, 5 min each time, and douching for 30 s. In the end the chip was dried by centrifugation; scanning, reading the data.

Altogether 10 strains of positive antibodies (with fluorescence intensity larger than 200) were screened from the chip, wherein 3 strains were verified by Western blotting, demonstrating that they can specifically recognize VEGF protein (VEGF verification data).

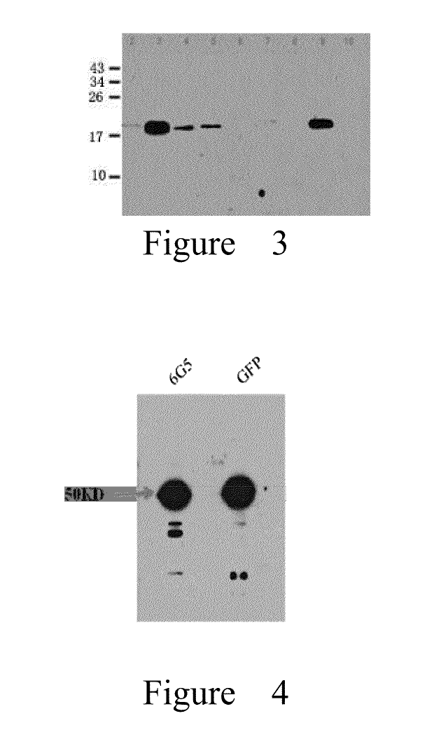

As shown in FIG. 3, lanes 3, 4, 5 were screened specific monoclonal antibodies, lane 9 was previously successfully verified positive antibody. It can be seen from the Western blotting results, the screened antibodies can specifically recognize VEGF protein, as did the known positive antibody.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

The claimed invention is:

1-126. (canceled)

127. A method for identifying an antibody to a target, which method comprises:
a) providing for an antibody library obtained from a mammal whose immune system has not been stimulated by a target exogenously, wherein said antibody library comprises less than 10⁷ different kinds of antibodies;
b) contacting said target with said antibody library under conditions suitable for binding between said target with an antibody in said antibody library, if such antibody existing in said antibody library; and
c) assessing binding between said target and said antibody to identify said antibody as an antibody to said target.

128. The method of claim 127, wherein one or more of the following A) through CC) apply:
A) the antibody library is produced by a mammal immunized with a plurality of polypeptides comprising different, random amino acid sequences;
B) the polypeptides does not comprise Cys, does not comprise 3 or more identical, consecutive amino acids, and/or does not comprise 5 or more identical amino acids;
C) the polypeptides are selected by the standards:
a) assigning an initial, identical score to all candidate polypeptides;
b) reducing the initial score by 1 point for each potential glycosylation site in a candidate polypeptide;
c) reducing the initial score by 4 points for each Lys or Arg residue in a candidate polypeptide; and
d) selecting candidate polypeptide with highest possible scores for the desired number of polypeptides for immunizing the mammal;
D) the mammal is immunized with at least 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different polypeptides;
E) the polypeptides comprise about 10, 11, 12, 13, 14, 15, 16, 17, 18, 19 or 20 amino acids;
F) the polypeptides comprise natural and/or non-natural amino acids, and/or are produced by chemical synthesis and/or recombinant production;
G) the polypeptides are selected from candidate polypeptides by the following standard:
a) each of the candidate polypeptides comprises about 10 amino acids that do not include Cys;
b) each of the candidate polypeptides does not comprise 3 or more identical, consecutive amino acids,
c) each of the candidate polypeptides does not comprise 5 or more identical amino acids;
d) each of the candidate polypeptides is assigned an initial score of 10;
e) reducing the initial score by 1 point for each potential glycosylation site in a candidate polypeptide;
f) reducing the initial score by 4 points for each Lys or Arg residue in a candidate polypeptide; and
g) selecting at least 10,000 candidate polypeptides with the highest scores;
H) the antibody library is produced by a mammal whose immune system has not been purposely stimulated, preferably selected from the group consisting of a mouse, a rat and a rabbit;
I) the antibody library is produced by a mammal immunized with an intact organism, an tissue, an cell or a whole protein extract of the organism, tissue or cell, and the intact organism, tissue, cell is immunologically distinct from the target;
J) (i) the intact organism is selected from the group consisting of an Arabidopsis thaliana, a mouse, a rat, a rabbit, a bovine, a goat, a Drosophila, a zebrafish, a Caenorhabditis elegans, rice and corn;
(ii) the tissue is selected from the group consisting of blood, Arabidopsis thaliana bud, Caenorhabditis elegans tissues at different developmental stages, and a mouse brain tissue; and/or
(iii) the cell is selected from the group consisting of a spleen cell, a tumor cell and a cell line, e.g., a human tumor cell line;
K) the antibody library:
a) is produced by a mammal immunized with a plurality of polypeptides comprising different, random amino acid sequences;
b) is produced by a mammal whose immune system has not been purposely stimulated;
c) is produced by a mammal immunized with an intact organism, an tissue, an cell or a whole protein extract of the organism, tissue or cell;
d) is produced by a mammal immunized with an antigen that is immunologically distinct from the target; or
e) a combination of any of a)-d);
L) the antibodies in the antibody library comprise polyclonal antibodies, monoclonal antibodies and/or hybridomas that produce monoclonal antibodies, and/or are affinity matured;
M) the antibody library comprises at least 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different antibodies;
N) at least some of the antibodies are immobilized on a solid surface;
O) all of the antibodies are immobilized on a solid surface;
P) the solid surface is a part of a biochip;
Q) the target is a polypeptide, e.g. selected from the group consisting of a linear polypeptide, a soluble polypeptide, a modified polypeptide, a toxic polypeptide and a polypeptide that causes autoimmunity in a subject;
R) the target is contacted with a subgroup of antibodies in the antibody library to determine if the subgroup of antibodies comprises an antibody that specifically binds to the target;
S) the subgroup of antibodies comprises an antibody that specifically binds to the target, and further comprising:
a) dividing the subgroup of antibodies into a smaller subgroup of antibodies; and
b) contacting the target to determine if the smaller subgroup of antibodies comprises an antibody that specifically binds to the target;
T) the steps a) and b) are repeated until an individual antibody that specifically binds to the target is identified;
U) which is used to identify antibodies that specifically bind to a plurality of the targets;
V) the antibody library is contacted with the plurality of the targets to identify antibodies or groups of antibodies that specifically bind to the plurality of the targets;
W) which further comprises contacting each of the plurality of the targets with the identified antibodies or groups of antibodies to identify antibodies or groups of antibodies that specifically bind to each of the plurality of the targets;
X) which further comprises:
a) dividing the identified antibodies or groups of antibodies into smaller subgroups of antibodies; and
b) contacting each of the plurality of the targets with the smaller subgroups of antibodies to determine if the smaller subgroup of antibodies comprises an antibody that specifically binds to each of the plurality of the targets;
Y) the steps a) and b) are repeated until an individual antibody that specifically binds to each of the plurality of the targets is identified;
Z) the successful rate for identifying an antibody that specifically binds to the target is at least 10%, 20%, 30%, 40%, 50%, 60%, 70%, 80%, 90%, 95%, or 100%;
AA) the antibody library comprises at least 10,000 different antibodies and the successful rate for identifying an antibody that specifically binds to the target is at least 80%, 85%, 90%, 95%, or 100%;
BB) the amino acid sequences of antibodies in the antibody library are unknown priori;
CC) the antibodies in the antibody library comprise intact (or complete) antibody molecules.

129. A product, which is one of the following products I) through):
I) an antibody that specifically binds to the target, wherein the antibody is identified by a method, which comprises:
a) providing for an antibody library obtained from a mammal whose immune system has not been stimulated by a target exogenously, wherein said antibody library comprises less than 10⁷ different kinds of antibodies;
b) contacting said target with said antibody library under conditions suitable for binding between said target with an antibody in said antibody library, if such antibody existing in said antibody library; and
c) assessing binding between said target and said antibody to identify said antibody as an antibody to said target;
II) an antibody library for identifying an antibody to a target, which antibody library is obtained from an mammal whose immune system has not been stimulated by a target exogenously, wherein said antibody library comprises less than 10⁷ different kinds of antibodies;
III) a polypeptide library, which polypeptide library comprises a plurality of isolated polypeptides comprising different, random amino acid sequences, wherein the polypeptides comprise about 10-20 amino acids, the polypeptides do not comprise Cys, do not comprise 3 or more identical, consecutive amino acids, and/or do not comprise 5 or more identical amino acids;
IV) an antibody library, which is produced by a method, comprising
a) immunizing a subject with the above polypeptide library of the product III);
b) recovering antibodies from said subject;
and optionally said method further comprising affinity purifying antibodies recovered from the subject using said product, which is III) the polypeptide library;
V) a polypeptide library, which polypeptide library comprises at least 10, 100, 1,000, 10,000, 15,000, 20,000, 25,000, 30,000, 35,000, 40,000, 45,000, 50,000, or all isolated polypeptides set forth in SEQ ID NOs: 1-54808;
VI) an antibody library, which is produced by a method, comprising
a) immunizing a subject with the above polypeptide library of the product V;
b) recovering antibodies from said subject;
and optionally said method further comprising affinity purifying antibodies recovered from the subject using said product, which is V) the polypeptide library;
VII) an antibody library, which antibody library comprises antibodies that specifically bind to at least 10, 100, 1,000, 10,000, 15,000, 20,000, 25,000, 30,000, 35,000, 40,000, 45,000, 50,000, or all polypeptides set forth in SEQ ID NOs: 1-54808;
VIII) an isolated antibody that specifically binds to Akt, which isolated antibody specifically binds to an epitope comprised in the amino acid sequence QDGGQKAVKD;
IX) an isolated antibody that specifically binds to ERK2, which isolated antibody specifically binds to an epitope comprised in the amino acid sequence HPLGSPGSAS;
X) an isolated antibody that specifically binds to Desmin, which isolated antibody specifically binds to an epitope comprised in the amino acid sequence REIRRYQKST;
XI) an isolated antibody that specifically binds to CBL4, which isolated antibody specifically binds to an epitope comprised in the amino acid sequence RSRARKQAYT;
XII) an isolated antibody that specifically binds to cholera toxin, which isolated antibody specifically binds to an epitope comprised in the amino acid sequence FEEREQANTA, EYQQAQLEAE or DSSMSMADSE;
XIII) an isolated antibody that specifically binds to VEGF, which isolated antibody specifically binds to an epitope comprised in the amino acid sequence VLDFILSMGL, AKRKAGTSPR or RNSDFSAGSP;
XIV) an antibody library, comprising:
(1) antibodies against random peptides with 10-20 amino acids,
(2) IgG antibodies, secreted by hybridoma cells produced from spleen cells of naïve mammal,
(3) IgG antibodies, secreted by hybridoma cells produced from spleen cells of mammal that are immunized by total protein extract, said protein extract is from a complete organism, one or more tissues thereof, and/or one or more cells thereof,
(4) IgG antibodies, secreted by stable hybridoma stains established against one or more antigens, or
(5) any combination of (1)-(4);
wherein said antibody library comprises at least 10000 different members, and said antibody library has a success rate of at least 85% when used for screening antibodies against proteins of interest;
XV) a combination, comprising the antibody library XIV from above;
XVI) a biochip, comprising the antibody library XIV from above.

130. A product according to claim 129, which is II) the antibody library, to which one or more of the following 1) through 5) apply:
1) a) is produced by a mammal immunized with a plurality of polypeptides comprising different, random amino acid sequences;
b) is produced by a mammal whose immune system has not been purposely stimulated;
c) is produced by a mammal immunized with an intact organism, an tissue, an cell or a whole protein extract of the organism, tissue or cell;
d) is produced by a mammal immunized with an antigen that is immunologically distinct from the target; or
e) a combination of any of a)-d);
2) which comprises at least 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different antibodies;
3) the antibodies in the antibody library comprise polyclonal antibodies, monoclonal antibodies and/or hybridomas that produce monoclonal antibodies, and/or are affinity matured;
4) the amino acid sequences of antibodies in the antibody library are unknown priori;
5) the antibodies in the antibody library comprise intact (or complete) antibody molecules.

131. A product according to claim 129, which is Ill) the polypeptide library, to which one or more of the following 1) through 2) apply:
1) the polypeptides are selected by the standards:
a) assigning an initial, identical score to all candidate polypeptides;
b) reducing the initial score by 1 point for each potential glycosylation site in a candidate polypeptide;
c) reducing the initial score by 4 points for each Lys or Arg residue in a candidate polypeptide; and
d) selecting candidate polypeptide with highest possible scores for the desired number of polypeptides;
2) which comprises at least 10,000 polypeptides.

132. A method for producing an antibody library, comprising
a) immunizing a subject with a polypeptide library of a product according to claim 129, which is III) the polypeptide library;
b) recovering antibodies from said subject;
and optionally said method further comprising affinity purifying antibodies recovered from the subject using said product, which is III) the polypeptide library;
and optionally, wherein a subject is immunized with a group of about 5-20 polypeptides in the library, and multiple subjects are immunized with multiple groups of about 5-20 polypeptides in the library, and the multiple groups of about 5-20 polypeptides encompass all the polypeptides in the library.

133. A product according to claim 129, which is IV) the antibody library, to which one or more of the following 1) through 2) apply:
1) which comprises at least 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different antibodies;
2) the antibodies in the antibody library comprise polyclonal antibodies, monoclonal antibodies and/or hybridomas that produce monoclonal antibodies, and/or are affinity matured.

134. A method for producing an antibody library, comprising
a) immunizing a subject with a polypeptide library of a product according to claim 129, which is V) the polypeptide library;
b) recovering antibodies from said subject;
and optionally said method further comprising affinity purifying antibodies recovered from the subject using said product, which is V) the polypeptide library.

135. A product according to claim 129, which is VI) the antibody library, to which one or more of the following 1) through 2) apply:
1) which comprises at least 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different antibodies;
2) the antibodies in the antibody library comprise polyclonal antibodies, monoclonal antibodies and/or hybridomas that produce monoclonal antibodies, and/or are affinity matured.

136. A product according to claim 129, which is VI) the antibody library, to which the following applies:
the antibodies in the antibody library comprise polyclonal antibodies, monoclonal antibodies and/or hybridomas that produce monoclonal antibodies, and/or are affinity matured.

137. A method for identifying a peptidic antigenic sequence to a target antibody, which method comprises:
a) contacting a target antibody with a polypeptide library comprising at least 10, 100, 1,000, 10,000, 15,000, 20,000, 25,000, 30,000, 35,000, 40,000, 45,000, 50,000, or all isolated polypeptides set forth in SEQ ID NOs: 1-54808 under conditions suitable for binding between said target antibody with a polypeptide in said polypeptide library, if such polypeptide existing in said polypeptide library; and
c) assessing binding between said target antibody and said polypeptide to identify a peptidic antigenic sequence to said target antibody.

138. The method of claim 137, to which one or more of the following 1) through 6) apply:
1) the target antibody is contacted with a subgroup of polypeptides in the polypeptide library to determine if the subgroup of polypeptides comprises a polypeptide that specifically binds to the target antibody;
2) the subgroup of polypeptides comprises a polypeptide that specifically binds to the target antibody, and further comprising
a) dividing the subgroup of polypeptides into a smaller subgroup of polypeptides; and
b) contacting the target antibody with the smaller subgroup of polypeptides to determine if the smaller subgroup of polypeptides comprises a polypeptide that specifically binds to the target antibody;
3) the steps a) and b) are repeated until an individual polypeptide that specifically binds to the target antibody is identified;
4) which is used to identify peptidic antigenic sequences to a plurality of target antibodies;
5) which further comprises isolating the polypeptide that specifically binds to the target antibody, and optionally determining amino acid sequence of the isolated polypeptide;
6) the target antibody is a biomarker, e.g. a diagnostic or prognostic biomarker.

139. A method for identifying a target associated with a condition, which method comprises either A) or B):
A) a) contacting a sample obtained from a source that has a condition with an antibody library, which is for identifying an antibody to a target, which antibody library is obtained from an mammal whose immune system has not been stimulated by a target exogenously, wherein said antibody library comprises less than 10⁷ different kinds of antibodies, and assessing binding, or a lack thereof, between a substance in said sample and an antibody in said antibody library;
b) contacting a sample obtained from a source that does not have said condition with an antibody library, which is for identifying an antibody to a target, which antibody library is obtained from an mammal whose immune system has not been stimulated by a target exogenously, wherein said antibody library comprises less than 10⁷ different kinds of antibodies, and assessing binding, or a lack thereof, between a substance in said sample and an antibody in said antibody library; and
c) identifying a substance as a target associated with said condition when there is a difference in said binding, or a lack thereof, between said substance and said antibody in steps a) and b);
B) a) contacting a sample obtained from a source that has a condition with a polypeptide library, which polypeptide library comprises a plurality of isolated polypeptides comprising different, random amino acid sequences, wherein the polypeptides comprise about 10-20 amino acids, the polypeptides do not comprise Cys, do not comprise 3 or more identical, consecutive amino acids, and/or do not comprise 5 or more identical amino acids, and assessing binding, or a lack thereof, between a substance in said sample and a polypeptide in said polypeptide library;
b) contacting a sample obtained from a source that does not have said condition with a polypeptide library, which polypeptide library comprises a plurality of isolated polypeptides comprising different, random amino acid sequences, wherein the polypeptides comprise about 10-20 amino acids, the polypeptides do not comprise Cys, do not comprise 3 or more identical, consecutive amino acids, and/or do not comprise 5 or more identical amino acids, and assessing binding, or a lack thereof, between a substance in said sample and a polypeptide in said polypeptide library; and
c) identifying a substance as a target associated with said condition when there is a difference in said binding, or a lack thereof, between said substance and said polypeptide in steps a) and b).

140. A method claim 139, which is A), to which one or more of the following 1) through 5) apply:
1) which is used for identifying a target associated with a condition in a subject, a target associated with a disease or disorder, or multiple targets associated with a condition;
2) the difference in the binding, or a lack thereof, between the substance and the antibody in steps a) and b) is qualitative or quantitative;
3) binding between the substance and the antibody in step a) and lack of the binding between the substance and the antibody in step b) identify the substance as a target associated with the condition;
4) lack of binding between the substance and the antibody in step a) and binding between the substance and the antibody in step b) identify the substance as a target associated with the condition;
5) the target comprises a polypeptide.

141. A method claim 139, which is B), to which one or more of the following 1) through 5) apply:
1) which is used for identifying a target associated with a condition in a subject, a target associated with a disease or disorder, or multiple targets associated with a condition;
2) the difference in the binding, or a lack thereof, between the substance and the polypeptide in steps a) and b) is qualitative or is quantitative;
3) binding between the substance and the polypeptide in step a) and lack of binding between the substance and the polypeptide in step b) identify the substance as a target associated with the condition;
4) wherein lack of binding between the substance and the polypeptide in step a) and binding between the substance and the polypeptide in step b) identify the substance as a target associated with the condition;
5) the target comprises an antibody.

142. A product according to claim 129, which is XIV) the antibody library, to which one or more of the following 1) through 5) apply:
1) which is in the form of hybridoma cell library;
2) said random peptides:
1) do not contain cysteine,
2) do not contain 3 or more consecutive same amino acids,
3) do not contain 5 or more same amino acids;
3) wherein the initial score of each random peptide is set as any value, and the random peptides are selected through the following process:
1) for amino acids with potential glycosylation site, each potential glycosylation site reduces one point from the score,
2) each amino acid K or R reduces 4 points from the score;
based on the above score, desired amount of peptides with highest score are selected from the top to the bottom;
4) (i) said naïve mammal is selected from mouse, rat, rabbit;
(ii) said complete organism is selected from Arabidopsis thaliana, mouse, rat, rabbit, cattle, caprine, Drosophila, zebrafish, threadworm, rice, or maize;
(iii) said one or more tissues are selected from blood tissue, Arabidopsis thaliana calyx, threadworm tissues in different developmental stages, or mouse brain tissue;
(iv) said one or more cells are selected from spleen cells, tumor cells or cell lines, such as human tumor cell lines;
5) the antibodies have been subjected to affinity maturation;
6) said antibody library comprises at least 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 different members.

143. A method for screening antibody against a protein of interest, comprising using the product according to claim 129, which is XIV) the antibody library, or a combination or biochip comprising said product XIV) the antibody library to screen one or more antibodies against said protein of interest.

144. The method according to claim 143, comprising one or more of the following 1) through 7):
1) (a) mixing said protein of interest with antibodies or antibody groups of said antibody library,
(b) selecting antibodies or antibody groups capable of binding said protein of interest;
2) (c) mixing said protein of interest with antibodies or antibody subgroups of the antibody groups selected in step (b),
(d) selecting antibodies or antibody subgroups capable of binding said protein of interest;
3) using the antibody subgroups selected in step (d) to repeat steps (c) and (d) until an antibody capable of binding said protein of interest is selected;
4) the screening is simultaneously conducted against several proteins of interest, comprising:
(a) mixing said several proteins of interest with antibodies or antibody groups of said antibody library,
(b) selecting antibodies or antibody groups capable of binding said several proteins of interest,
(c) mixing each of said several proteins of interest, separately, with the antibodies or antibody groups capable of binding said several proteins of interest selected in step (b), and then respectively selecting antibodies or antibody groups capable of binding each of said several proteins of interest;
5) (d) mixing each of said several proteins of interest, separately, with antibodies or antibody subgroups of the antibody groups selected in step (c) capable of binging the respective protein of interest,
(e) respectively selecting antibodies or antibody subgroups capable of binding each of said several proteins of interest;
6) using the antibody subgroups selected in step (e) to repeat steps (d) and (e) until antibodies capable of binding each said protein of interest are respectively selected;
7) said protein of interest is post-translational modified protein or polypeptide, or toxic protein or polypeptide.
